(19) United States
(12) Patent Application Publication
(10) Pub. No.: US 2025/0195669 A1
(43) Pub. Date: Jun. 19, 2025

(54) FOLATE-ANTIBODY CONJUGATES AS LYSOSOME TARGETING DEGRADERS

(71) Applicant: Wisconsin Alumni Research Foundation, Madison, WI (US)

(72) Inventors: Weiping Tang, Middleton, WI (US); Yaxian Zhou, Madison, WI (US); Chunrong Li, Madison, WI (US); Hao Wu, Madison, WI (US)

(73) Assignee: Wisconsin Alumni Research Foundation, Madison, WI (US)

(21) Appl. No.: 18/848,741

(22) PCT Filed: Mar. 22, 2023
(86) PCT No.: PCT/US2023/015896

§ 371 Date: Sep. 19, 2024

Related U.S. Application Data
(60) Provisional application No. 63/322,426, filed on Mar. 22, 2022.

(51) Int. Cl. A61K 47/55; A61K 47/68

(52) U.S. Cl. CPC A61K 47/551; A61K 47/6849

(57) ABSTRACT

Provided herein are bifunctional lysosomal targeting degraders that comprise folate or a folate analog configured to bind to folate receptor as a shuttle molecule for lysosome degradation, and a protein binder configured to bind a membrane or extracellular protein of interest. The bifunctional degraders find use, e.g., for selectively targeted degradation of membrane and extracellular proteins via the endosomal/lysosomal pathway. Also provided herein are compositions comprising the bifunctional degraders, as well as methods of using the bifunctional degraders.

### CROSS-REFERENCE TO RELATED APPLICATIONS

Priority is hereby claimed to provisional application Ser. No. 63/322,426, filed Mar. 22, 2022, the content of which is incorporated herein by reference.

### FEDERAL FUNDING STATEMENT

This invention was made with government support under GM120357 awarded by the National Institutes of Health. The government has certain rights in the invention.

### BACKGROUND

Targeted Protein Degradation (TPD) is emerging as an exciting therapeutic option to confront diseases involving aberrantly expressed or mutated disease-causing proteins by engaging our body's natural protein disposal systems. TPD by chimeric molecules is a novel therapeutic modality (Deshaies, 2020, Nature. 580:329-338). These chimeras are heterobifunctional molecules with one end binding to the protein of interest (POI) and the other end directing the resulting complex towards a certain degradation pathway. PROteolysis TArgeting Chimera (PROTAC) has received the most attention to date. (See Sakamoto et al., 2001, Proc. Natl. Acad. Sci. 98:8554-8559; Luh et al., 2020, Angew. Chem. Int. Ed. 59:15448-15466; Wu et al., 2020, Nat. Struct. Mol. Biol. 27:605-614.) PROTACs contain an E3 ligase ligand to route the targeted protein to the proteasome for degradation. (Lai and Crews, 2017, Nat. Rev. Drug Discovery. 16:101-114; Salami and Crews, 2017, Science. 355:1163-1167; Cromm and Crews, 2017, Cell Chem. Biol. 24:1181-1190; Toure and Crews, 2016, Angew. Chem. Int. Ed. 55:1966-1973.) However, PROTACs are only capable of depleting intracellular proteins. There are many disease targets that are membrane or extracellular proteins.

To broaden the scope of targets, researchers have reported a way of tagging extracellular protein targets with a ligand for membrane receptors involved in active transport of molecules into the cell. The tagged protein is naturally shuttled to the lysosome in the cell where it is degraded. The bifunctional lysosome targeting degraders are generally created by conjugating ligands of the lysosome targeting receptors (LTRs) on the cell surface with ligands that can bind to the extracellular protein target. This process is shown schematically in FIG. 1. The LTRs employed in the past studies are carbohydrate binding proteins including cation-independent mannose 6-phosphate receptor (CIM6PR or insulin-like growth factor-II receptor) (Banik et al., 2020, Nature. 584:291-297) and asialoglycoprotein receptor (ASGPR) (Zhou et al., 2021, ACS Cent. Sci. 7:499-506; Ahn et al., 2021, Nat. Chem. Biol. 17:937-946; Caianiello et al., 2021, Nat. Chem. Biol. 17:947-953). The receptor-ligand interaction triggers the internalization of the extracellular proteins through receptor-mediated endocytosis, further inducing the degradation of the targets in the lysosome.

One type of bifunctional lysosome targeting degraders were developed by conjugating the ligand of the CIM6PR on the cell surface with a molecule that binds to the extracellular protein target (Banik et al., 2020, Nature. 584:291-297). This type of bifunctional lysosome-targeting degraders that recruit CIM6PR was also termed LYsosome TArgeting Chimeras (LYTACs). CIM6PR is expressed ubiquitously in most cell types. The receptor-ligand interaction triggers the internalization of the extracellular proteins through receptor-mediated endocytosis, further inducing the degradation of the targets in the lysosome. CIM6PR is a transmembrane receptor that transports proteins bearing N-glycans capped with mannose 6-phosphate (M6P) residues to lysosomes (Ghosh et al., 2003, Nat. Rev. Mol. Cell Biol. 4:202-213; Coutinho et al., 2012, Mol. Genet. Metab. 105:542-550). Early studies showed that albumin modified with M6P increased the cellular uptake (Beljaars et al., 1999, Hepatology. 29:1486-1493). Subsequently, CIM6PR was used to deliver therapeutic drugs conjugated with M6P derivatives for lysosomal enzyme replacement therapy and cancer treatment (Ghosh et al., 2003, Nat. Rev. Mol. Cell Biol. 4:202-213; Gary-Bobo et al., 2007, Curr. Med. Chem. 14: 2945-2953). Various molecules, such as peptides, proteins or liposome, were covalently linked to the M6P or its analogues to achieve targeted drug delivery (Hoogendoorn et al., 2014, Angew. Chem. Int. Ed. 53:10975-10978; Crucianelli et al., 2014, RSC Adv. 4:58204-58207; Das et al., 2016, Acs Macro Letters. 5:809-813; Agarwal et al., 2016, Chem. Commun. 52:327-330; Hyun et al., 2018, Cell Chem. Biol. 25:1255-1267). To extend the usage of CIM6PR/M6P system to targeted protein degradation, LYTAC was constructed by conjugating a mixture of polyglycopeptides containing 20-40 units of M6P analogues to the antibody of POI. Different from drug delivery process, which involves the internalization of a covalent linked M6P-protein target, LYTAC allows the trafficking of a complex formed by the non-covalent interaction between the protein target and LYTAC. It was shown that LYTAC could successfully degrade both secreted and membrane proteins in the lysosome through CIM6PR (Banik et al., 2020, Nature. 584:291-297). However, the challenge associated with the synthesis and attachment of a heterogenous mixture of polymeric glycopeptides with 20-40 units of M6P analogues to antibodies employed in the LYTAC system limited its utility in drug development. In addition, since CIM6PR is ubiquitously expressed in most cell types, the POI is delivered to all cell types non-selectively. There remains an unmet need for selective means of degrading extracellular proteins.

### SUMMARY

Provided herein is a bifunctional lysosomal targeting degrader, comprising a folate moiety or an analog thereof configured to bind to folate receptor as a shuttle molecule for lysosome degradation, and a protein-binding moiety configured to bind a membrane or extracellular protein.

In one version, the protein-binding moiety of the bifunctional degrader is configured to bind a membrane protein. The membrane protein may be a membrane receptor. For example, the membrane receptor may be (by way of example and not limitation) epidermal growth factor receptor (EGFR). The membrane protein may be an immune inhibitory receptor or a ligand of an immune inhibitory receptor, such as CD47. The membrane protein may also be an immune checkpoint molecule. For example, the immune checkpoint molecule may be the programmed cell death ligand 1 (PD-L1).

In another version, the protein-binding moiety of the bifunctional degrader is configured to bind an extracellular protein. The extracellular protein may be (by way of example and not limitation) a ligand for a membrane receptor, an auto-antibody, a secreted protein, a mutated protein, or the like.

The protein-binding moiety of the bifunctional degrader may be any type of moiety capable of binding to the membrane or extracellular protein to be targeted for degradation via the endosomal/lysosomal pathway. In certain aspects, the protein-binding moiety is selected from a polypeptide, a ligand, an aptamer, a nanoparticle, and a small molecule.

In one version, the protein-binding moiety of the bifunctional degrader is a small molecule.

In another version, the protein-binding moiety of the bifunctional degrader is a polypeptide.

The protein-binding moiety can be an antibody, either a whole antibody or a fragment of an antibody wherein the fragment retains protein-binding activity. The antibody or fragment thereof is preferably configured to bind to a membrane receptor, a ligand of membrane receptor, an immune inhibitory receptor, a ligand of an immune inhibitory receptor, an immune checkpoint molecule, an auto-antibody, a secreted protein, or a mutated protein. In a specific version of the bifunctional lysosomal targeting degrader, the antibody is configured to bind to an EGFR protein. In one embodiment, the antibody is Cetuximab. In another embodiment, the antibody is configured to bind to a CD47 protein. The antibody may also be configured to bind to a PD-L1 protein. In one embodiment, the antibody is Atezolizumab.

The bifunctional degrader may further comprise a linker connecting the folate moiety or analog thereof to the protein-binding moiety. Preferred linkers include poly(ethylene glycol) (“PEG”)), N-hydroxysuccinimide (“NHS”), and the like, either alone or in combination.

By binding to folate receptor, the bifunctional lysosomal targeting degrader disclosed herein selectively targets cells that express folate receptors. This includes cancer cells and other neoplastic cells that express folate receptors.

Also provided herein is a pharmaceutical composition that includes any of the bifunctional lysosomal targeting degraders of the present disclosure. The bifunctional lysosomal targeting degraders may be administered neat, or in combination with a pharmaceutically acceptable carrier configured for any chosen route of administration (e.g., enteral, parenteral, topical, rectal, and the like).

Also provided herein is a method of degrading a membrane or extracellular protein, comprising contacting the membrane or extracellular protein with any of the bifunctional lysosomal targeting degraders of the present disclosure, wherein the bifunctional lysosomal targeting degrader shuttles the membrane or extracellular protein to the lysosome for degradation.

Also provided herein is a method that comprises administering to an individual in need thereof a therapeutically effective, neoplastic cell growth-inhibiting amount of any of the pharmaceutical compositions of the present disclosure. In some embodiments, the individual is a human. In some embodiments, the individual has a cancer.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 shows chimeric degrader-induced degradation of extracellular protein of interest (POI) through lysosome targeting receptor (LTR).

FIG. 2A shows chemical structure of FA-PEGn-NHS. FIG. 2B shows chemical structures of DBCO-PEG3-NHS and N3-PEGn-FA.

FIG. 3 is a series of three graphs showing fluorescence intensity indicating uptake of NA-650 in Hela cells (left-hand graph), HepG2 cells (center graph), and MCF7 cells (right-hand graph) treated with FA-PEG3-biotin (“NA-650+FA-biotin”) or without FA-PEG3-biotin (“NA-650”).

FIG. 4A shows MALDI-MS spectra of anti-mouse IgG Fab fragment with folate labeling (“Fab-FA”) or without folate labeling (“Fab”).

FIG. 4B shows MALDI-MS spectra of full-size anti-mouse IgG with folate labeling (“Ab-FA”) or without folate labeling (“Ab”).

FIGS. 5A and 5B show gel fluorescence analysis of mouse anti-biotin-IgG-647 protein uptake by Hela cells treated with folate labeled antibodies (“Ab-FA” and “Fab-FA”) or antibodies alone (“Ab” and “Fab”). FIG. 5C is a histogram of the relative fluorescent intensity of the gel bands shown in FIG. 5B.

FIG. 6 shows gel fluorescence analysis of mouse anti-biotin-IgG-594 protein uptake by A549, HepG2, FaDu, and Hela cells treated with folate-labeled antibody (“Ab-FA”) or the antibody alone (“Ab”).

FIG. 7 shows MALDI-MS spectra of Ctx, Ctx-FA1, Ctx-FA2, and Ctx-FA3.

FIG. 8 shows western blot of EGFR in Hela cells treated with Ctx, Ctx-FA1 and Ctx-FA2 at 3, 10, and 30 nM for 48 h (upper panel) and 72 h (lower panel). Actin is used as loading control. “-” represents negative controls.

FIG. 9 shows western-blot of EGFR in Hela cells treated with Ctx and Ctx-FA at 0.1, 1, and 10 nM for 6 h and 48 h. Actin is used as loading control. “-” represents negative controls.

FIG. 10 shows western blot of EGFR in multiple cancer cell lines treated with Ctx and Ctx-FA (“degrader”) at 10 nM for 48 h. Actin is used as loading control. “-” represents negative controls.

FIG. 11 shows western blot of EGFR and LTR (folate receptor) in multiple cell lines treated with Ctx-FA at 10 nM for 48 h. Actin is used as loading control.

FIG. 12 shows normalization of EGFR and LTR to actin based on the gel image of FIG. 11.

FIG. 13 shows percentage of EGFR degradation and LTR/EGFR ratio in multiple cell lines calculated based on the data of FIG. 12.

FIG. 14 shows western blot of phosphorylated EGFR (pEGFR) and MAPK (pMAPK) in cells pretreated with 10 nM Ctx-FA (“degrader”) for 24 h followed by 10 min or 60 min incubation with 100 ng/ml EGF. Actin is used as loading control. “-” represents negative controls.

FIG. 15 shows western blot of EGFR in HepG2 liver cells treated with Ctx, Ctx-oligometric M6P, Ctx-tri-GalNAc (GN) and Ctx-FA. Actin is used as loading control.

FIG. 16 shows western blot of EGFR, p-EGFR, pAkt, and pMAPK in Cal27 head and neck cancer cells treated with M6P-Ctx and Ctx-FA at 10 and 300 nM. Actin is used as loading control. “-” represents negative controls.

FIG. 17 shows western blot of EGFR, p-EGFR, pAkt, and pMAPK in FaDu head neck cancer cells treated with M6P-Ctx and Ctx-FA at 10 and 300 nM. Actin is used as loading control. “-” represents negative controls.

FIGS. 18A and 18B show western blots of EGFR, pAkt, and pMAPK in Hela cells treated with M6P-Ctx and Ctx-FA at 0, 0.1, 1, 10 and 100 nM (FIG. 18A) and at 0, 2, 4, 6, 8, 16, 24, 48 and 72 h (FIG. 18B). Actin is used as loading control.

FIG. 19 shows western blot of EGFR in FaDu cells (upper panel) and HepG2 cells (lower panel) co-treated with 10 nM Ctx-FA (“degrader”) and 200 nM bafilomycin (“BAF”) or 3 mM folic acid (“ligand”) for 6 h, compared to cells treated with Ctx-FA (“degrader”) alone. Actin is used as loading control. “-” represents negative controls.

FIGS. 20A and 20B show confocal microscopy imaging of EGFR and marker for lysosome membrane (LAMP1) in FaDu head neck cancer cells. FIG. 20A shows two views of the same well of FaDu cells treated with 10 nM Ctx-FA for 24 h. FIG. 20B shows comparison of FaDu cells treated with 10 nM Ctx-FA degrader for 24 h (upper panel) and not treated with the Ctx-FA degrader (lower panel).

FIG. 21 shows MALDI-MS spectra of Atz with folate labeling (“Atz-FA”) or without folate labeling (“Atz”).

FIG. 22 shows western blot of PD-L1 in multiple cancer cell lines treated with 10 nM Atz-FA (“degrader”) or Atz alone (“Atz”) for 24 h. Actin is used as loading control. “-” represents negative controls.

FIG. 23 shows western blot of CD47 in HepG2 and MDA-MB-231 cells treated with 10 nM folate labeled CD47 antibody (“anti-CD47-degrader”) or the antibody alone (“anti-CD47”). Actin is used as loading control. “-” represents negative controls.

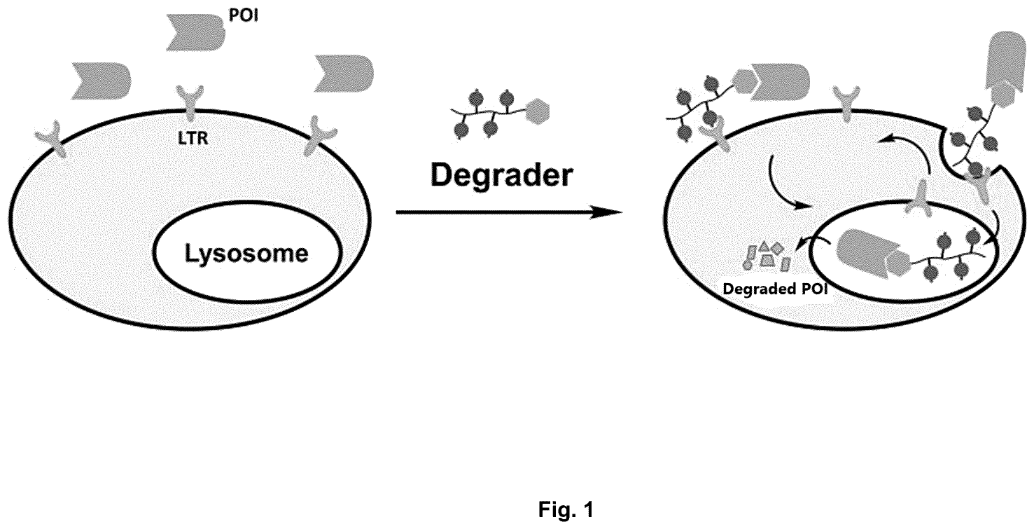

### DETAILED DESCRIPTION

Provided herein are bifunctional lysosome targeting degraders that comprise: (a) a folate moiety or folate analog that is configured to bind to folate receptor as a shuttle molecule for lysosome degradation; and (b) a protein-binding moiety (or herein referred to as a “protein binder”) that is configured to bind a membrane or extracellular protein of interest. The bifunctional degrader disclosed herein finds use, e.g., for selectively targeted degradation of membrane and extracellular proteins via the endosomal/lysosomal pathway. In one aspect, the bifunctional degraders induce degradation of oncogenic proteins specifically in cancer cells through the folate receptor, which is overexpressed in many cancer cells. Also provided herein are compositions comprising the bifunctional degraders, as well as methods of using the bifunctional degraders to inhibit disease states, including cancers.

It is to be understood that the bifunctional degraders, compositions, and methods disclosed herein are not limited to particular embodiments described, as such may, of course, vary. It is also to be understood that the terminology used herein is for the purpose of describing particular embodiments only and is not intended to be limiting.

It is noted that, as used herein and in the appended claims, the singular forms “a”, “an”, and “the” include plural referents unless the context clearly dictates otherwise. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

It is appreciated that certain features of the bifunctional degraders, compositions, and methods, which are, for clarity, described in the context of separate embodiments, may also be provided in combination in a single embodiment. Conversely, various features of the bifunctional degraders, compositions, and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present bifunctional degraders, compositions, and methods and are disclosed herein just as if each and every such sub-combination was individually and explicitly disclosed herein. It is also noted that the bifunctional degraders, compositions, and methods provided herein suitably may be practiced in the absence of any element which is not specifically disclosed herein.

### Bifunctional Lysosome Targeting Degraders

Provided herein are bifunctional lysosome targeting degraders that include folate or its analogs that is configured to bind to folate receptor as a shuttle molecule for lysosome degradation, operationally linked to a protein-binding moiety (“protein binder”) that is configured to bind a membrane or extracellular protein of interest. As used herein, “operationally linked” means that the folate moiety (or folate analog moiety) may be linked directly or indirectly to the protein binder. Thus a direct link would comprise a direct chemical bond between the two moieties (without any intervening atoms) or there may be a linking moiety (“linker”) of any description between the folate moiety (or folate analog moiety) and the protein binding moiety.

Folic acid is a small molecule that crosses the cell membrane through binding to the folate receptor. As disclosed herein, folic acid is attached to a binder of a membrane or extracellular protein of interest creating a folate-protein binding degrader. Once inside the cell, folic acid enters the endosome where it deposits the protein of interest payload, and that protein of interest is degraded.

The term “folate analog” is used herein to mean a moiety that is structurally analogous to folate and which is configured to bind to folate receptor. The term “folate analog,” includes, but is not limited to, folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analogs or derivatives thereof. For example, the deaza analogs include the 1-deaza, 3-deaza, 5-deaza, 8-deaza, and 10-deaza analogs of folate. The dideaza analogs include, for example, 1,5-dideaza, 5,10-dideaza, 8,10-dideaza, and 5,8-dideaza analogs of folate. Other folate analogs useful as complex-forming ligands include the folate receptor-binding analogs aminopterin, amethopterin (methotrexate), N10-methylfolate, 2-deamino-hydroxyfolate, deaza analogs such as 1-deazamethopterin or 3-deazamethopterin, and 3′,5′-dichloro-4-amino-4-deoxy-N10-methylpteroylglutamic acid (dichloromethotrexate). These folic acid analogs are conventionally termed “folates,” reflecting their ability to bind with folate-receptors. Additional analogs of folic acid that bind to folic acid receptors are described in U.S. Patent Application Publication Nos. 2005/0227985 and 2004/0242582, which are incorporated herein by reference.

Folate receptors, or folate binding proteins, include single chain glycoproteins that bind and contribute to the uptake of folates and other compounds in vivo (Elwood, 1989, J. Biol. Chem. 264:14893-14901). Certain folate receptors are single-chain glycoproteins with a high affinity binding site for folate and other compounds such as methotrexate. The mature folate receptor glycoprotein has a size of about 42 kDa and has been observed to participate in the internalization of folates and antifolates into cells (Elwood et al., 1997, Biochemistry 36:1467-1478). The folate receptor gene family includes four members: FRa or FOLR1, FRB or FOLR2, FRY or FOLR3, and FR8 or FOLR4 (Leamon and Jackman, 2008, Vitam Horm.

79:203-33).

Folate receptor alpha (FRa) is a glycosylphosphatidylinositol linked cell-surface glycoprotein that has high affinity for folates. Except for low levels in kidney and lung, most normal tissues do not express FRa. But high levels of FRa have been found in epithelial ovarian cancer, endometrial adenocarcinoma, non-small cell lung carcinoma (NSCLC) of the adenocarcinoma subtype, and triple-negative breast cancer (TNBC) (Scaranti et al., 2020, Nat. Rev. 17:349-359). FRa expression is maintained in metastatic foci and recurrent carcinomas in ovarian cancer patients, and FRa expression has been observed after chemotherapy in epithelial ovarian and endometrial cancers. Folate receptor beta (FRB) was originally thought to exist only in placenta, but it is also detected in myeloid cells and acute myelogenous leukemias (Pan et al., 2002, Blood. 100:594-602). Due to the restricted expression of folate receptors, the bifunctional degraders disclosed herein impart selectivity of cells that express folate receptors to degrade membrane and extracellular proteins of interest.

As disclosed herein, the bifunctional lysosome targeting degraders include a protein binder that is configured to bind a membrane or extracellular protein of interest. In some embodiments, the protein binder binds a membrane protein.

In certain embodiments, the membrane protein is a membrane receptor. Membrane receptors of interest include, but are not limited to, stem cell receptors, immune cell receptors, growth factor receptors, cytokine receptors, hormone receptors, receptor tyrosine kinases, a receptor in the epidermal growth factor receptor (EGFR) family (e.g., HER2 (human epidermal growth factor receptor 2), etc.), a receptor in the fibroblast growth factor receptor (FGFR) family, a receptor in the vascular endothelial growth factor receptor (VEGFR) family, a receptor in the platelet derived growth factor receptor (PDGFR) family, a receptor in the rearranged during transfection (RET) receptor family, a receptor in the Eph receptor family, a receptor in the discoidin domain receptor (DDR) family, and a mucin protein (e.g., MUC1).

In a specific version, the membrane receptor is EGFR, which is known to be frequently mutated or overexpressed in different types of human cancers (Yarden and Pines, 2012, Nat Rev Cancer. 12:553-563; Sigismund et al., 2018, Mol. Oncol. 12:3-20).

The membrane protein may be an immune inhibitory receptor. As used herein, an “immune inhibitory receptor” is a receptor present on an immune cell that negatively regulates an immune response. Examples of inhibitory immune receptors include immune inhibitory receptors of the Ig superfamily, including but not limited to: CD200R, CD300a (IRp60; mouse MAIR-I), CD300f (IREM-1), CEACAMI (CD66a), FcγRIIb, ILT-2 (LIR-1; LILRB1; CD85j), ILT-3 (LIR-5; CD85k; LILRB4), ILT-4 (LIR-2; LILRB2), ILT-5 (LIR-3; LILRB3; mouse PIR-B); LAIR-1, PECAM-1 (CD31), PILR-α (FDF03), SIRL-1, and SIRP-α. Further examples of immune inhibitory receptors include sialic acid-binding Ig-like lectin (Siglec) receptors, e.g., Siglec 7, Siglec9, and/or the like. Additional examples of immune inhibitory receptors include C-type lectins, including but not limited to: CLEC4A (DCIR), Ly49Q and MICL. Details regarding immune inhibitory receptors may be found, e.g., in Steevels et al., 2011, Eur. J. Immunol. 4:575-587.

The membrane protein may optionally be a ligand of an immune inhibitory receptor, one example of which is CD47, which binds to SIRP-α to prevent phagocytosis and known to be overexpressed in cancer cells (Eladl et al., 2020, J. Hematol. Oncol. 13:96).

The membrane protein may also be an immune checkpoint molecule including immune checkpoint proteins and ligands. Non-limiting examples of immune checkpoint molecules include PD-1, PD-L1, CTLA4, TIM3, LAG3, TIGIT, and a member of the B7 family. In one embodiment, the membrane protein is PD-L1 (Programmed Cell Death Ligand 1), which binds PD-1 (programmed cell death-1) to inhibit apoptosis and known to be overexpressed in cancer cells (Yi et al., 2021, J. Hematol. Oncol. 14:10).

As disclosed herein, the bifunctional lysosome targeting degraders include a protein binder that is configured to bind a membrane or extracellular protein of interest. In some embodiments, the protein binder binds an extracellular protein.

The extracellular protein may be a ligand for a membrane receptor. Membrane receptor ligands of interest include, but are not limited to, growth factors (e.g., epidermal growth factor (EGF), vascular endothelial growth factor (VEGF), and the like), cytokines (e.g., an interleukin, an interferon, a tumor necrosis factor (TNF), a transforming growth factor β (TGF-β), including any particular subtypes of such cytokines), hormones, and the like.

Alternatively, the extracellular protein may be an antibody, such as an antibody that binds a membrane protein or a different extracellular protein. The antibody may be an auto-antibody. By “auto-antibody” is meant an antibody produced by the immune system that is directed against one or more of the individual's own proteins. Cancer cells can induce an immunological response resulting in the production of tumor-associated auto-antibodies. Non-limiting examples of auto-antibodies include rheumatoid factor (RF), antinuclear antibody (ANA), antineutrophil cytoplasmic antibodies (ANCA), anti-double stranded DNA (anti-dsDNA), anticentromere antibodies (ACA), anticyclic citrullinated peptide antibodies (anti-CCP), extractable nuclear antigen antibodies (ENA), anticardiolipin antibodies, beta-2 glycoprotein 1 antibodies, antiphospholipid antibodies (APA), lupus anticoagulants (LA), anti-tissue transglutaminase (anti-tTG), anti-gliadin antibodies (AGA), intrinsic factor antibodies, parietal cell antibodies, thyroid antibodies, smooth muscle antibodies (SMA), antimitochondrial antibodies (AMA), anti-glomerular basement membrane (GBM), acetylcholine receptor (AChR) antibodies, etc.

The extracellular protein may be a secreted protein, including, but not limited to, secreted growth factors, extracellular matrix-degrading proteinases, cell motility factors and immunoregulatory cytokines or other bioactive molecules.

The extracellular protein may also be a mutated protein.

When the protein binder of the bifunctional degrader binds a membrane or extracellular protein, the membrane or extracellular protein may be present on a cancer cell or produced by a cancer cell. By “cancer cell” is meant a cell exhibiting a neoplastic cellular phenotype, which may be characterized by one or more of, for example, abnormal cell growth, abnormal cellular proliferation, loss of density-dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell,” “neoplastic cell,” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like. In some embodiments, the membrane protein present on the cancer cell is a tumor-associated antigen or a tumor-specific antigen.

The protein binder of the bifunctional degrader may be any type of moiety capable of binding to the membrane or extracellular protein to be targeted for degradation via the endosomal/lysosomal pathway. In certain aspects, the protein binder is selected from a polypeptide, a ligand (e.g., a ligand for a membrane receptor, where the membrane receptor is targeted for degradation), an aptamer, a nanoparticle, and a small molecule.

The protein binder may be a small molecule. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In one embodiment, the small molecule is biotin.

The protein binder may be a polypeptide, such as an antibody. The terms “antibody” and “immunoglobulin” include antibodies or immunoglobulins of any isotype (e.g., IgG (e.g., lgG1, lgG2, lgG3 or lgG4), IgE, IgD, IgA, IgM, etc.); whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to the membrane or extracellular protein, including, but not limited to, Fv, single chain Fv (scFv), Fab, F(ab′) 2, Fab′, (scFv′) 2, diabodies, and nanobodies; chimeric antibodies; monoclonal antibodies; fully human antibodies; humanized antibodies (e.g., humanized whole antibodies, humanized antibody fragments, etc.); and fusion proteins including an antigen-binding portion of an antibody and a non-antibody protein or fragment thereof. The antibodies may be detectably labeled, e.g., with an in vivo imaging agent, or the like. The antibodies may be further conjugated to other moieties, such as, e.g., polyethylene glycol (PEG), etc. Fusion to an antibody Fc region (or a fragment thereof), conjugation to PEG, etc. may find use, e.g., for increasing serum half-life of the antibody upon administration to the subject.

In certain versions, the antibody is configured to bind to a cancer antigen.

The antibody may also be configured to bind to an intact complement or a fragment thereof. In certain embodiments, the antibody binds to one or more immunodominant epitope(s) within intact complement or a fragment thereof.

Alternatively, the antibody may bind to a membrane receptor or a membrane receptor ligand.

Or the antibody may bind to an epidermal growth factor (EGF) protein, e.g., a human EGF, or one or more immunodominant epitope(s) within an EGF protein.

In certain embodiments, the antibody binds to an EGFR protein. In certain embodiments, the antibody binds to one or more immunodominant epitope(s) within an EGFR protein. In a certain embodiment, the antibody comprises the CDRs present in Cetuximab (Ctx). In another certain embodiment, the antibody comprises the variable light chain and variable heavy chain present in Cetuximab. In a particular embodiment, the antibody is Cetuximab.

In certain embodiments, the antibody binds to an immune inhibitory receptor. In certain embodiment, the antibody binds to one or more immunodominant epitope(s) within an immune inhibitory receptor.

In certain embodiments, the antibody binds to a ligand of an immune inhibitory receptor. In certain embodiment, the antibody binds to one or more immunodominant epitope(s) within a ligand of an immune inhibitory receptor. In certain embodiments, the antibody binds to a CD47 protein. In certain embodiments, the antibody binds to one or more immunodominant epitope(s) within a CD47 protein.

In certain embodiments, the antibody binds to an immune checkpoint molecule. In certain embodiments, the antibody binds to one or more immunodominant epitope(s) within an immune checkpoint molecule. In certain embodiments, the antibody binds to a PD-L1 protein. In certain embodiments, the antibody binds to one or more immunodominant epitope(s) within PD-L1 protein. In a certain embodiment, the antibody comprises the CDRs present in Atezolizumab (Atz). In another certain embodiment, the antibody comprises the variable light chain and variable heavy chain present in Atezolizumab. In a particular embodiment, the antibody is Atezolizumab.

The bifunctional lysosome targeting degraders disclosed herein may be in any suitable format. In some embodiments, the bifunctional degrader is a conjugate. Accordingly, in certain embodiments, a bifunctional degrader disclosed herein includes folate conjugated to the protein binder. In certain embodiments, one or more linkers may be employed to facilitate conjugation of folate to the protein binder. Non-limiting examples of such linkers include ester linkers (e.g., N-hydroxysuccinimide (NHS) ester, sulfo-NHS ester or PFP ester or thioester), amide linkers, maleimide or maleimide-based linkers; valine-citrulline linkers; hydrazone linkers; N-succinimidyl-4-(2-pyridyldithio) butyrate (SPDB) linkers; Succinimidyl-4-(A/-maleimidomethyl) cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof. In one embodiment, the linker is PEG. In another embodiment, the linker is PEG and NHS.

In certain aspects, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. According to certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry and Stump, 2010, Bioconjugate Chem. 21:5-13.

The folate-protein binder conjugates can be formed by covalently linking folate to the protein binder, either directly or through one or more linker molecule, through one or more functional groups to form a covalent conjugate. The Example section below shows exemplary methods of preparing folate and antibody conjugates.

In certain aspects, the bifunctional degrader enhances degradation of the membrane or extracellular protein relative to degradation of the membrane or extracellular protein in the presence of the protein binder alone. According to some embodiments, the bifunctional degrader enhances degradation of the membrane or extracellular protein relative to degradation of the membrane or extracellular protein in the presence of folate or the protein binder alone. By “enhances degradation” in this context means the membrane or extracellular protein is degraded in the presence of the bifunctional degrader and is not degraded in the presence of the protein binder alone, or the presence of folate or the protein binder alone, under the same conditions; or the membrane or extracellular protein is degraded in the presence of the bifunctional degrader to a greater extent than the membrane or extracellular protein is degraded in the presence of the protein binder alone, or the presence of folate or the protein binder alone, under the same conditions. When the membrane or extracellular protein is degraded in the presence of the bifunctional degrader to a greater extent than the membrane or extracellular protein is degraded in the presence of the protein binder alone, or the presence of folate or the protein binder alone under the same conditions, the degradation may be 1.2 fold or greater, 1.4 fold or greater, 1.6 fold or greater, 1.8 fold or greater, 2 fold or greater, 2.5 fold or greater, 3 fold or greater, 3.5 fold or greater, 4 fold or greater, 4.5 fold or greater, 5 fold or greater, 5.5 fold or greater, 6 fold or greater, 6.5 fold or greater, 7 fold or greater, 7.5 fold or greater, 8 fold or greater, 8.5 fold or greater, 9 fold or greater, 9.5 fold or greater, or 10 fold or greater in the presence of the bifunctional degrader.

### Compositions

Disclosed herein are compositions that include any of the bifunctional lysosomal targeting degraders in the present disclosure.

The compositions may optionally include a bifunctional degrader of the present disclosure present in a liquid medium. The liquid medium may be an aqueous liquid medium, such as water, a buffered solution, and the like. One or more additives such as a salt (e.g., NaCl, MgCl2, KCl, MgSO4), a buffering agent (a Tris buffer, N-(2-Hydroxyethyl) piperazine-N′-(2-ethanesulfonic acid) (HEPES), 2-(NMorpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.), a protease inhibitor, glycerol, and the like may be present in such compositions.

Also disclosed herein are pharmaceutical compositions that include any of the bifunctional lysosomal targeting degraders of the present disclosure, and a pharmaceutically acceptable carrier. The pharmaceutical compositions generally include a therapeutically effective amount of the bifunctional degrader. By “therapeutically effective amount” is meant a dosage sufficient to produce a desired result, e.g., an amount sufficient to effect beneficial or desired therapeutic (including preventative) results, such as a reduction in cellular proliferation in an individual having a cell proliferative disorder (e.g., cancer) associated with the membrane or extracellular protein to which the protein binder of the bifunctional degrader binds, etc. An effective amount may be administered in one or more administrations.

A bifunctional degrader of the present disclosure can be incorporated into a variety of formulations for therapeutic administration. More particularly, the bifunctional degrader can be formulated into pharmaceutical compositions by combination with appropriate pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the bifunctional degraders of the present disclosure suitable for administration to an individual (e.g., suitable for human administration) are generally sterile and may further be free of detectable pyrogens or other contaminants contraindicated for administration to an individual according to a selected route of administration.

In pharmaceutical dosage forms, the bifunctional degrader can be administered alone or in appropriate association, as well as in combination, with other pharmaceutically active compounds. The following methods and excipients are merely examples and are in no way limiting.

For oral preparations, the bifunctional degrader can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The bifunctional degraders can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical composition may be in a liquid form, a lyophilized form or a liquid form reconstituted from a lyophilized form, where the lyophilized preparation is to be reconstituted with a sterile solution prior to administration. The standard procedure for reconstituting a lyophilized composition is to add back a volume of pure water (typically equivalent to the volume removed during lyophilization); however, solutions comprising antibacterial agents may be used for the production of pharmaceutical compositions for parenteral administration.

An aqueous formulation of the bifunctional degrader may be prepared in a pH buffered solution, e.g., at pH ranging from about 4.0 to about 8.0, such as from about 4.5 to about 7.5, e.g., from about 5.0 to about 7.0. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

### Method of Use

Disclosed herein are methods of using the bifunctional lysosomal targeting degraders of the present disclosure.

Provided herein are methods of degrading a membrane or extracellular protein. Such methods include contacting the membrane or extracellular protein with any of the bifunctional lysosomal targeting degraders of the present disclosure, under conditions in which the bifunctional lysosomal targeting degrader shuttles the membrane or extracellular protein to lysosome for degradation. Such methods find use in a variety of applications. In certain aspects, the method is performed in vitro (e.g., in a tube, cell culture plate or well, or the like) and finds use, e.g., in testing and/or research applications. In other aspects, the method is performed in vivo (e.g., in an individual to whom the bifunctional degrader is administered) and finds use, e.g., in clinical/therapeutic applications.

Also provided are methods that include administering to an individual in need thereof a therapeutically effective amount of any of the bifunctional degraders or any of the pharmaceutical compositions of the present disclosure. A variety of individuals are treatable according to the subject methods. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human.

An effective amount of the bifunctional degrader (or pharmaceutical composition including same) is an amount that, when administered alone (e.g., in monotherapy) or in combination (e.g., in combination therapy) with one or more additional therapeutic agents, in one or more doses, is effective to reduce the symptoms of a medical condition of the individual (e.g., cancer) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the bifunctional degrader or pharmaceutical composition.

The methods include administering to an individual having cancer a therapeutically effective amount of any of the bifunctional degraders or any of the pharmaceutical compositions of the present disclosure. According to such methods, the protein binder of the bifunctional degraders binds a membrane or extracellular protein that at least contributes to the individual's cancer, and where targeted degradation of the membrane or extracellular protein using the bifunctional degrader treats the individual's cancer. In certain aspects, the protein binder binds to a protein selected from a membrane receptor, a ligand for a membrane receptor, an immune inhibitory receptor, a ligand of an immune inhibitory receptor, an immune checkpoint molecule, an autoantibody, a secreted protein, and a mutated protein.

For example, the individual to be treated may have a cancer characterized by the presence of a solid tumor, a semi-solid tumor, a primary tumor, a metastatic tumor, or the like. In some embodiments, the individual has a cancer selected from breast cancer, melanoma, lung cancer, colorectal cancer, prostate cancer, glioma, bladder cancer, endometrial cancer, kidney cancer, leukemia (e.g., acute myeloid leukemia (AML)) liver cancer (e.g., hepatocellular carcinoma (HCC), such as primary or recurrent HCC), non-Hodgkin lymphoma, pancreatic cancer, thyroid cancer, any combinations thereof, and any sub-types thereof.

In any of the methods of using the bifunctional degraders of the present disclosure, the bifunctional degrader generally enhances degradation of the membrane or extracellular protein relative to degradation of the membrane or extracellular protein in the presence of the protein binder alone. Similarly, in any of the methods of using the bifunctional degraders of the present disclosure, according to some embodiments, the bifunctional degrader enhances degradation of the membrane or extracellular protein relative to degradation of the membrane or extracellular protein in the presence of folate or the protein binder alone.

By “treat”, “treating” or “treatment” is meant at least an amelioration of the symptoms associated with the medical condition (e.g., cell proliferative disorder, e.g., cancer) of the individual, where amelioration is used in a broad sense to refer to at least a reduction in the magnitude of a parameter, e.g., symptom, associated with the medical condition being treated. As such, treatment also includes situations where the medical condition, or at least symptoms associated therewith, are completely inhibited, e.g., prevented from happening, or stopped, e.g., terminated, such that the individual no longer suffers from the medical condition, or at least the symptoms that characterize the medical condition.

The bifunctional degrader or pharmaceutical composition may be administered to the individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. In some embodiments, the administering is by parenteral administration. Routes of administration may be combined, if desired, or adjusted depending upon the bifunctional degrader and/or the desired effect. The bifunctional degraders or pharmaceutical compositions may be administered in a single dose or in multiple doses. In some embodiments, the bifunctional degrader or pharmaceutical composition is administered intravenously. In some embodiments, the bifunctional degrader or pharmaceutical composition is administered by injection, e.g., for systemic delivery (e.g., intravenous infusion) or to a local site.

### EXAMPLES

### Methods

Antibody Labeling with FA-PEGn-NHS:

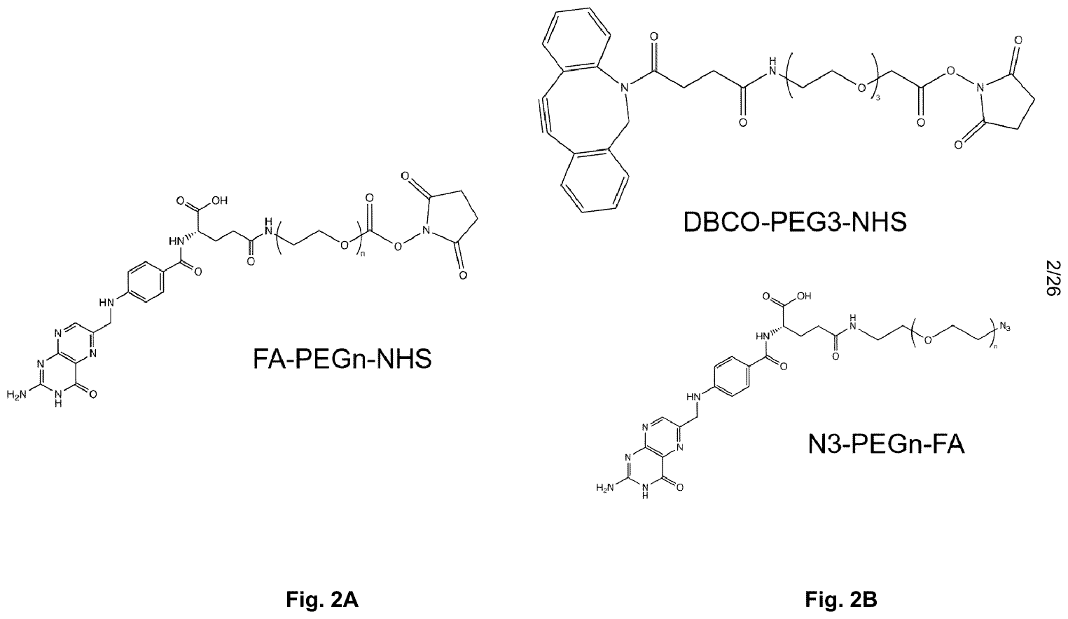

To label the antibody with folate (FA), 100 μL of the antibody (concentration at 1.8 mg/mL) in PBS was mixed with folate-NHS ester at 1:25 molar ratio. The reaction was incubated overnight at room temperature on a rotator, followed by filtration with 500 μL of PBS for 5 times using 10 kDa Amicon Centrifugal Filter. See FIG. 2A for the chemical structure of FA-PEGn-NHS.

Antibody Labeling with DBCO-PEG3-NHS and N3-PEGn-FA:

To label the antibody with DBCO, 200 μL of the antibody (concentration at 1.8 mg/mL) in PBS was mixed with DBCO-PEG3-NHS ester at 1:25 molar ratio. The reaction was incubated overnight at room temperature on a rotator, followed by filtration with 500 μL of PBS for 5 times using 10 kDa Amicon Centrifugal Filter. Then, the concentration of DBCO-labeled antibody was determined by BCA assay and mixed with N3-PEGn-FA at 1:25 molar ratio. The reaction was incubated overnight at room temperature on a rotator, followed by filtration with 500 μL of PBS for 5 times using 10 kDa Amicon Centrifugal Filter. See FIG. 2B for chemical structures of DBCO-PEG3-NHS and N3-PEGn-FA.

### MALDI-MS

α-Cyano-4-hydroxycinnamic acid (HCCA) was dissolved in 50% acetonitrile/water to give a 10 mg/mL solution as the matrix solution. The sample was absorbed on Omix C4 pipette tips, washed by 0.1% TFA for three times and then eluted with 20 μL 75% acetonitrile/water. 1 μL sample solution and 1 μL HCCA solution were spotted on the MALDI target plate and mixed thoroughly before the spot was allowed to dry under room temperature. MALDI-MS spectra were acquired on Bruker UltraFlex MALDI-TOF/TOF mass spectrometer operated in linear positive ion mode. Masses were calculated from windowed raw data in Sigmaplot 13.0 by fitting to gaussian curves, with constant baseline as an additional free parameter. Parameter starting values were the default values of the program, and were automatically iterated 200 times to obtain fits. Plots were made in Origin 2020, where high-frequency noise was removed using 100 points windowed FFT filter.

### Mouse IgG Uptake Experiment:

Cells were plated at 70% confluence in a 24-well plate. Complete growth media supplemented with 50 nM of mouse anti-biotin-IgG-647 and 25 nM of FA labelled goat anti-mouse IgG was sequentially added. The cells were incubated at 37° C. for 24 h and then lysed for in gel fluorescence analysis.

### Targeted Protein Degradation:

Cells were seeded at 70% confluence in a 24-well plate. Next day, cells were treated with 10 nM degrader for 24 or 48 h before collection for western blot analysis. For EGFR degradation, 100 ng/ml EGF was added and incubated for 10 or 60 min after 24 h degradation to stimulate the EGFR signaling pathway.

### Western Blotting:

Cells were lysed in 1×RIPA lysis buffer containing 25 mM Tris, pH 7-8, 150 mM NaCl, 0.1% (w/v) sodium dodecyl sulfate (SDS), 0.5% sodium deoxycholate, 1% (v/v) Triton X-100, protease inhibitor cocktail (Roche, one tablet per 10 mL) and 1 mM phenylmethylsulfonyl fluoride on ice for 10 min. The lysates were then centrifuged at 16 000 g at 4° C. for 15 min and the supernatant was collected followed by measuring the protein concentration using BCA assay. Lysates were adjusted to the equal amount before mixed with the 4× Laemmli Loading Dye and heated at 99° C. for 5 min. After cooling down, samples were loaded onto 7.5% or 12% SDS-polyacrylamide gel electrophoresis and transferred to PVDF membrane. The membrane was first blocked in 5% (w/v) nonfat milk in the TBS-T washing buffer (137 mM NaCl, 20 mM Tris, 0.1% (v/v) Tween) and then incubated with primary antibodies at 4° C. overnight. After 3 washes with TBST, the membrane was incubated with secondary HRP-linked antibodies for 1 h, and then washed 3 times with TBST. Then the membrane was incubated in the Clarity ECL substrate for 3-5 min before acquiring the immunoblot by ChemiDoc MP Imaging Systems.

### Confocal Microscopy:

FaDu cells were seeded onto 8-well chamber slides at the density of 20,000 cells/well in 200 μL of complete culture medium. After adhesion, cells were treated with 10 nM of Ctx-FA for 24 h at 37° C. Cells were then washed with PBS for 3 times and fixed with 4% paraformaldehyde for 15 min followed by permeabilization with 0.5% Triton-100 for 5 min. After blocking with 5% BSA for 1 h at RT, the cells were co-incubated with anti-EGFR antibody and anti-LAMP1 antibody or EEAl antibody in 1% BSA overnight at 4° C. The next day, cells were washed with PBS and then incubated with anti-mouse-488 or anti-rabbit-594 secondary antibody for 1 h at RT. Then the cells were mounted with slowfade-antifade mounting medium containing DAPI. Images were acquired by Leica SP8 3×STED super-resolution microscope at 100× magnification with a 10× eyepiece and analyzed by ImageJ.

### Competition Assay:

FaDu or HepG2 cells were seeded at at 70% confluence in a 24-well plate. Next day, cells were co-treated with 10 nM degrader and 200 nM Bafilomycin A1 or 3 mM folic acid for 6 h before collection for western blot analysis.

### Example 1: Uptake Mediated by Small Molecule Degrader and Antibody-Based Degrader

In this example, we tested a small molecule degrader folate-PEG3-biotin ((Nanocs) with the following chemical structure:

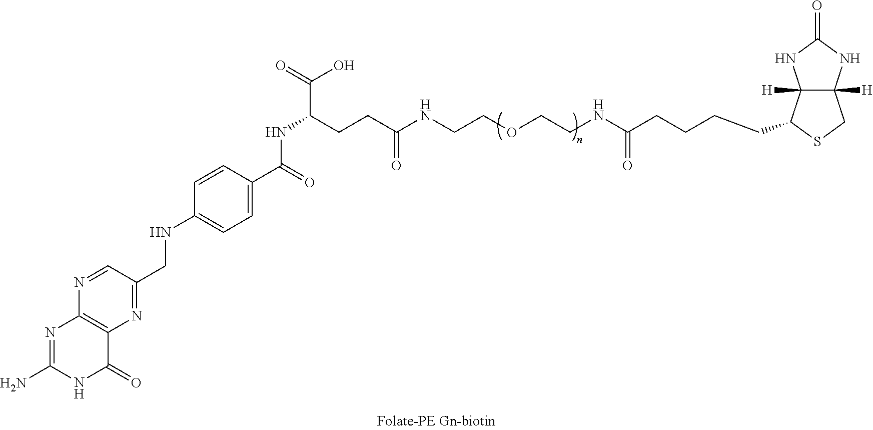

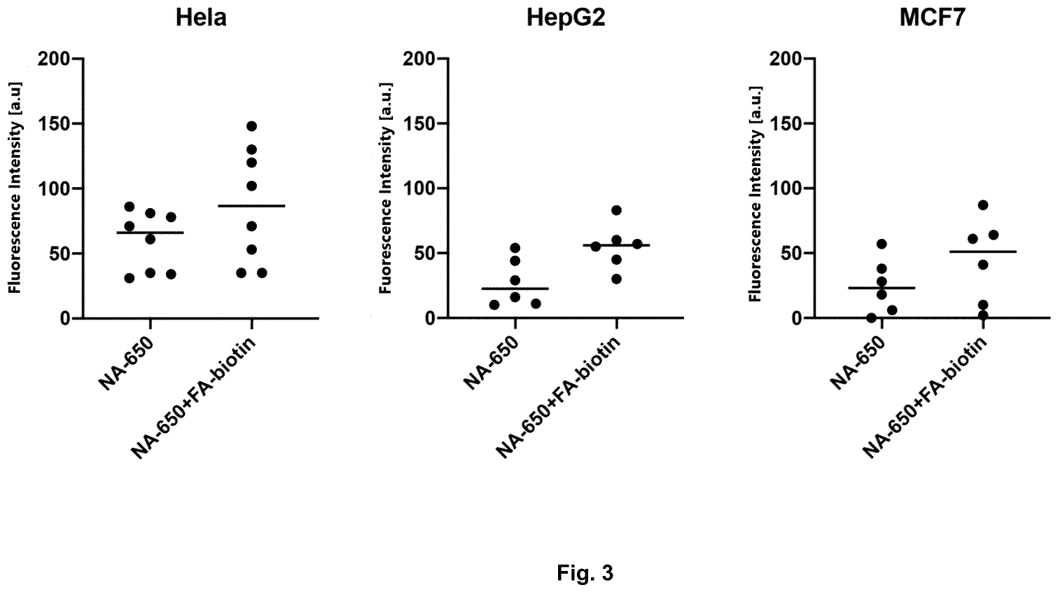

The Folate-PEG3-biotin degrader includes biotin as the protein binder, which binds to avidin and streptavidin with high specificity and affinity. By treating Hela, HepG2 and MCF7 cells with or without the folate-PEG3-biotin degrader, we observed higher uptake of NA-650 (NeutrAvidin-650, a fluorescently labeled protein to which biotin strongly binds) in cells treated with the degrader compared to cells treated with NA-650 alone (FIG. 3).

Next, we tested antibody-based degraders that includes an antibody as the protein binder, and the antibody is labeled with Folate-PEG10-NHS which has the following chemical structure:

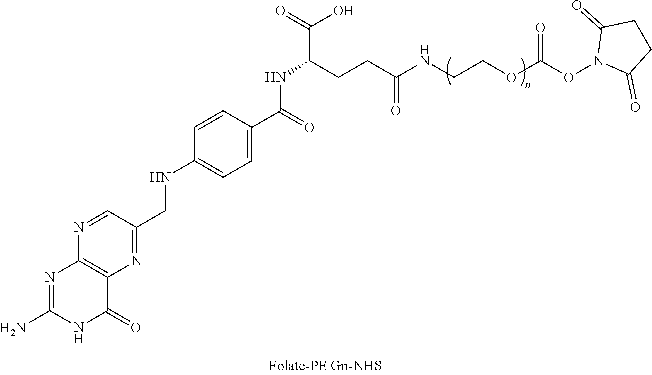

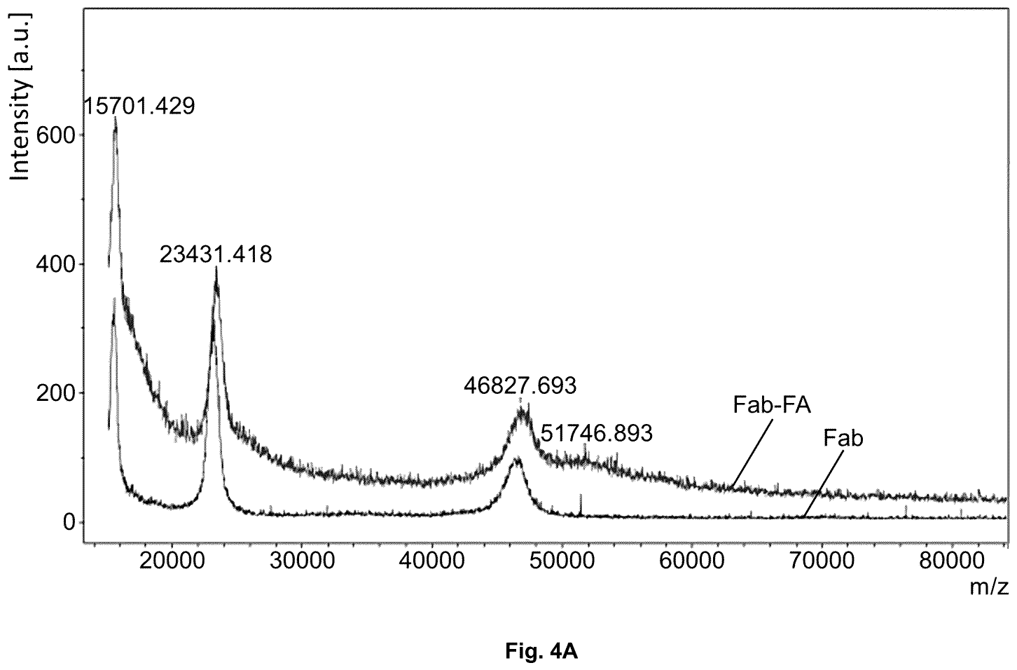

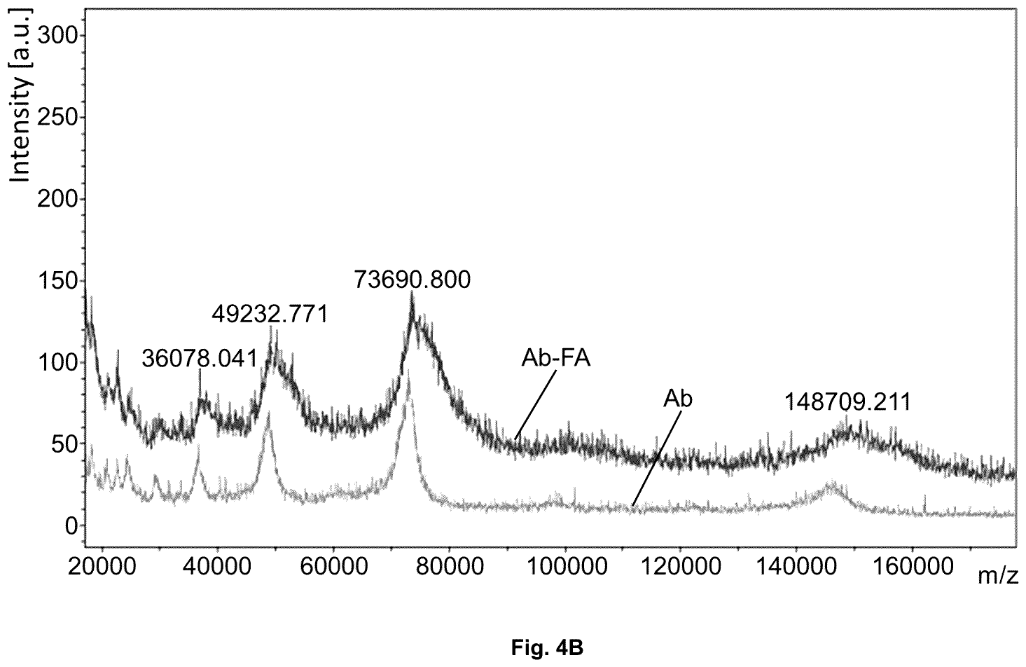

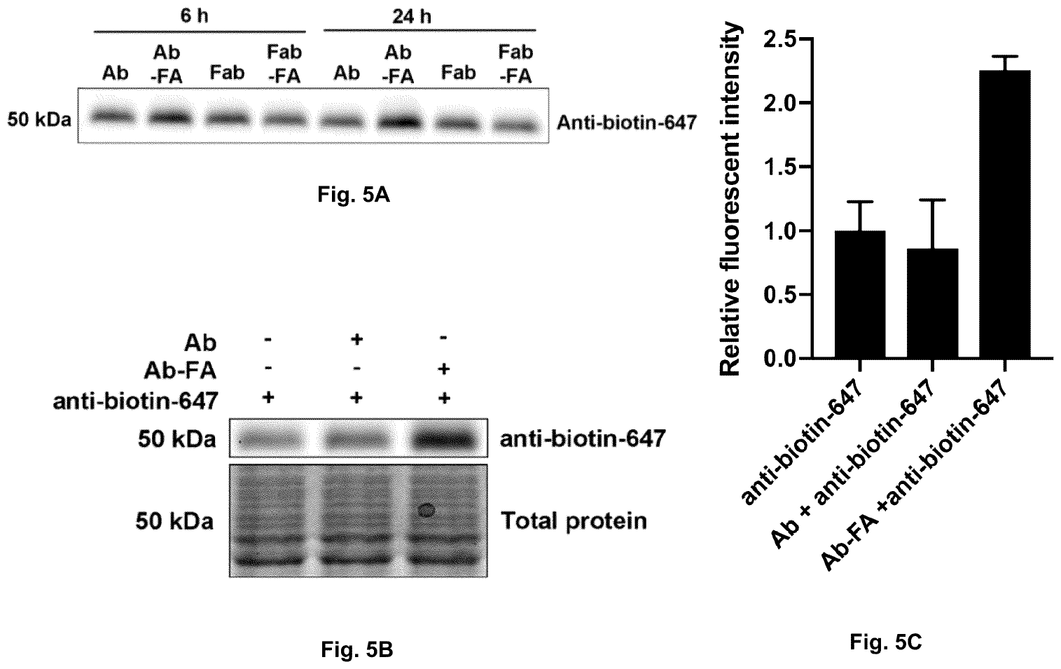

We labeled anti-mouse IgG antibody (Ab 150 kDa, Fab 50 kDa) with folate-PEG10-NHS and monitored uptake of fluorescent protein target mouse anti-biotin-IgG-647 in Hela cells treated with or without the folate labeled antibody. FIGS. 4A and 4B show MALDI-MS spectra of the anti-mouse IgG Fab fragment and the full-size anti-mouse IgG with or without folate labeling. FIGS. 5A-5C show gel fluorescence analysis indicating higher uptake of the protein targets in cells treated with the folate labeled anti-mouse IgG antibody compared to cells treated with the antibody alone. Uptake of the protein targets is higher in Hela cells treated with the folate labeled full-size anti-mouse IgG than the anti-mouse IgG Fab monomers

(FIG. 5A).

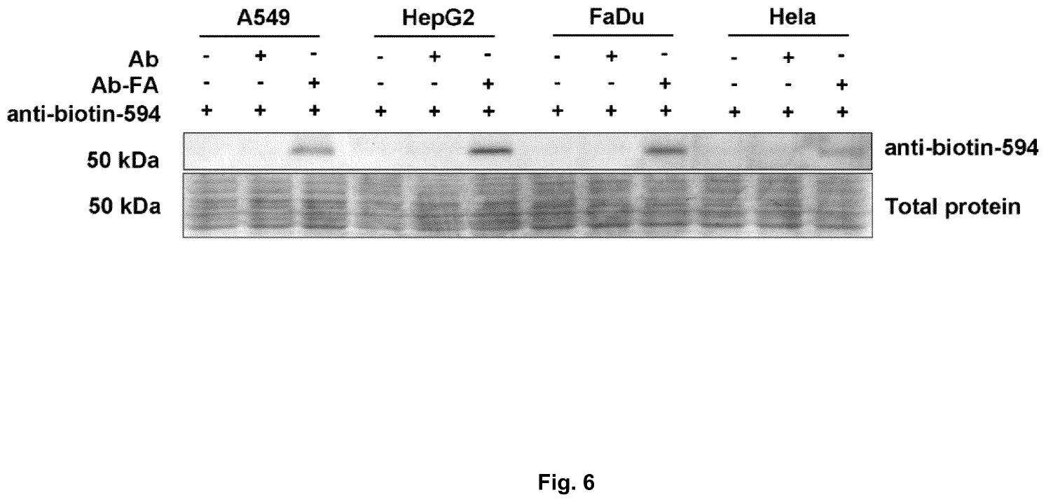

In general, uptake mediated by small molecule degrader is moderate compared to antibody-based degrader. Consistent uptake was observed with the treatment of antibody-based degrader for 24 h in Hela cells (FIGS. 5B and 5C). Uptake of anti-biotin-IgG-594 protein by the antibody-based degrader was also observed in multiple cell lines as shown in FIG. 6.

### Example 2: EGFR Degradation

Cetuximab (Ctx) is an approved antibody drug that blocks the function of epidermal growth factor receptor (EGFR) on the cell surface for the treatment of metastatic colorectal cancer and head and neck cancer. (Ctx is available commercially from ImClone LLC, New York, New York, under the registered trademark “ERBITUX”®; U.S. NDC Codes 66733-948-23 and 66733-958-23.) The major side effects of Ctx are associated with the blocking of EGFR in normal tissues. Selectively degrading EGFR in cancer cells can offer significant therapeutic benefits.

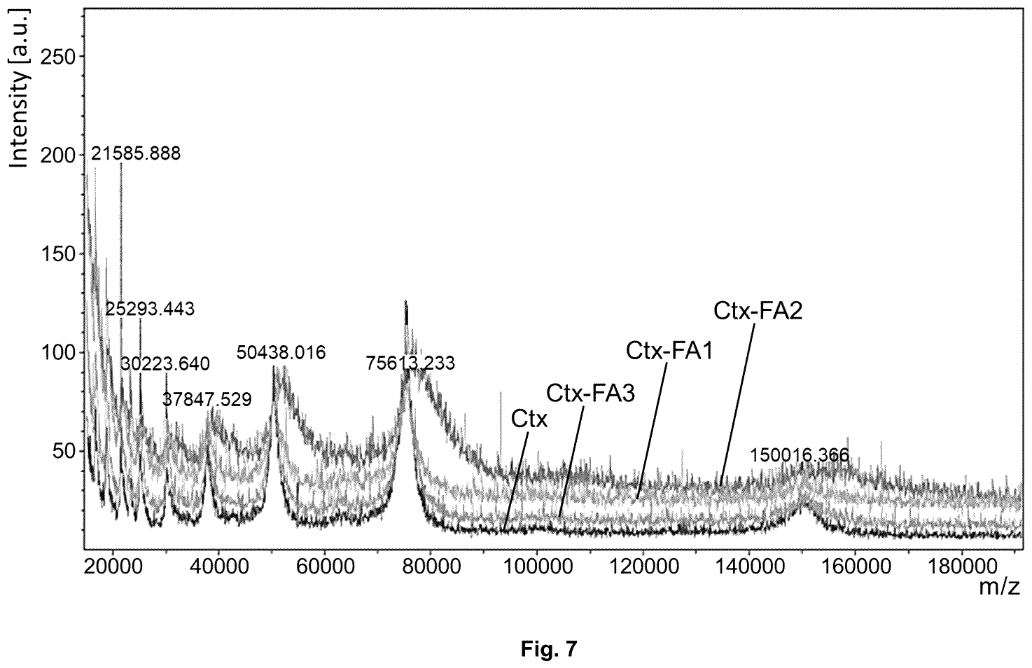

In this example, we labeled Ctx with folate-PEG10-NHS from two different vendors (Nanocus and Ruixibiotech) to generate degraders Ctx-FA1 and Ctx-FA2. We also labeled Ctx with folate-PEG3-NHS to generate degraders Ctx-FA3. FIG. 7 shows MALDI-MS spectra of Ctx, Ctx-FA1, Ctx-FA2, and Ctx-FA3.

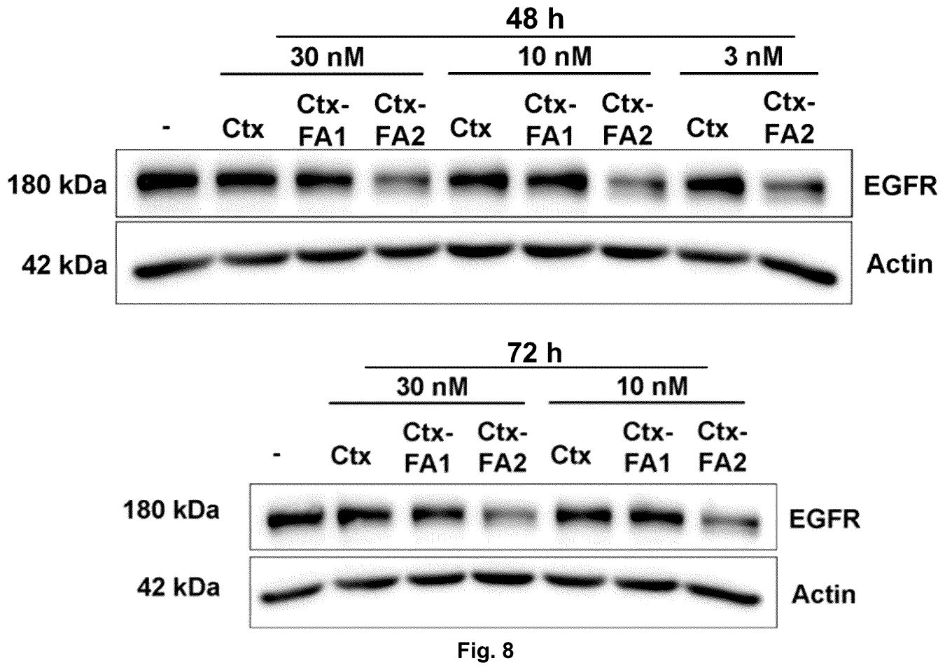

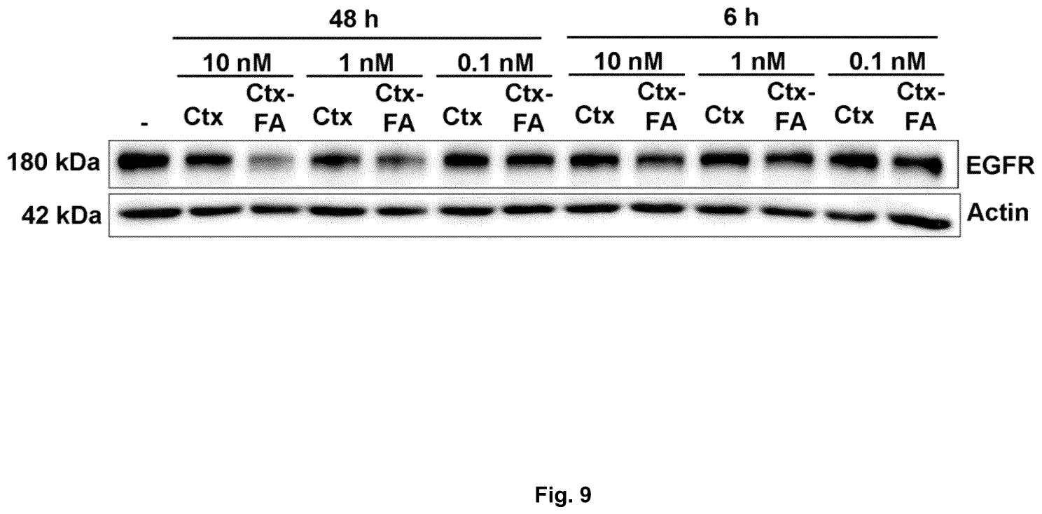

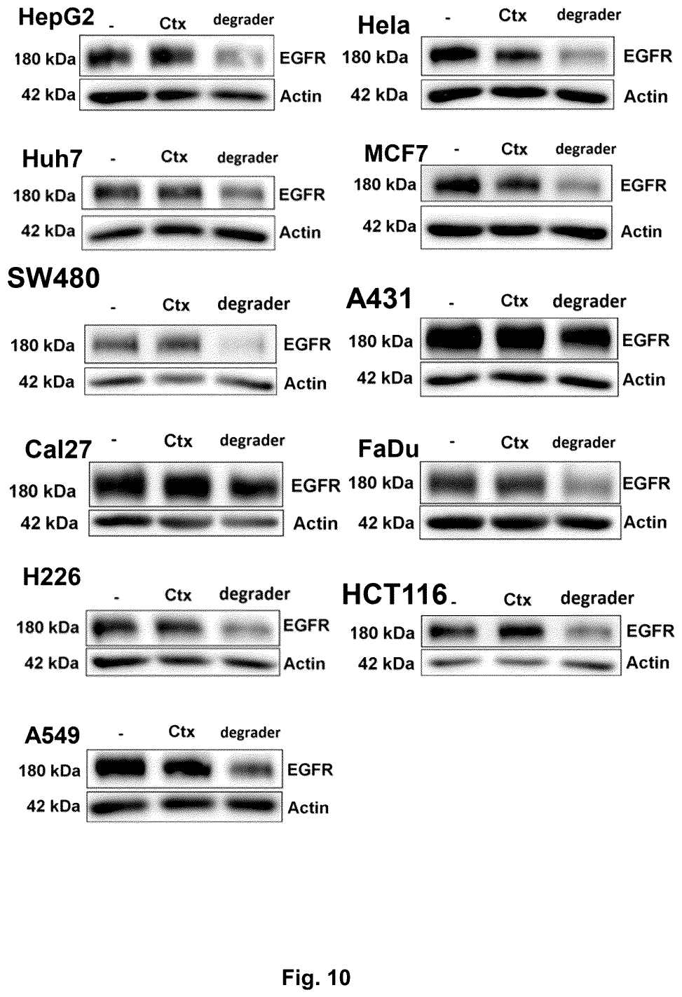

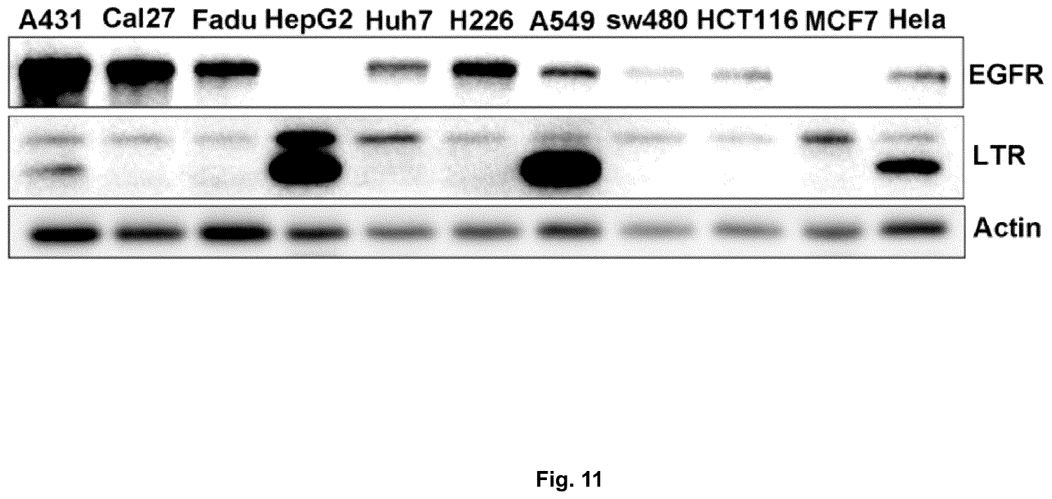

Our results indicate that Ctx-FA2 could induce the degradation of EGFR in Hela cells with 48 h and 72 h treatment, as shown in FIG. 8. The results are reproducible with different concentrations of the Ctx-FA degrader as shown in FIG. 9. Degradation of EGFR was also observed in multiple cancer cell lines treated by the Ctx-FA degrader as shown in FIG. 10.

Compared with uptake of anti-biotin protein targets in the media, we observed more efficient degradation of membrane protein EGFR by the Ctx-FA degrader. This is just the opposite compared to our previous studies on GalNAc labeled antibodies in which uptake of protein targets in the media is much more efficient than the degradation of EGFR (Zhou et al., 2021, ACS Cent. Sci. 7:499-506).

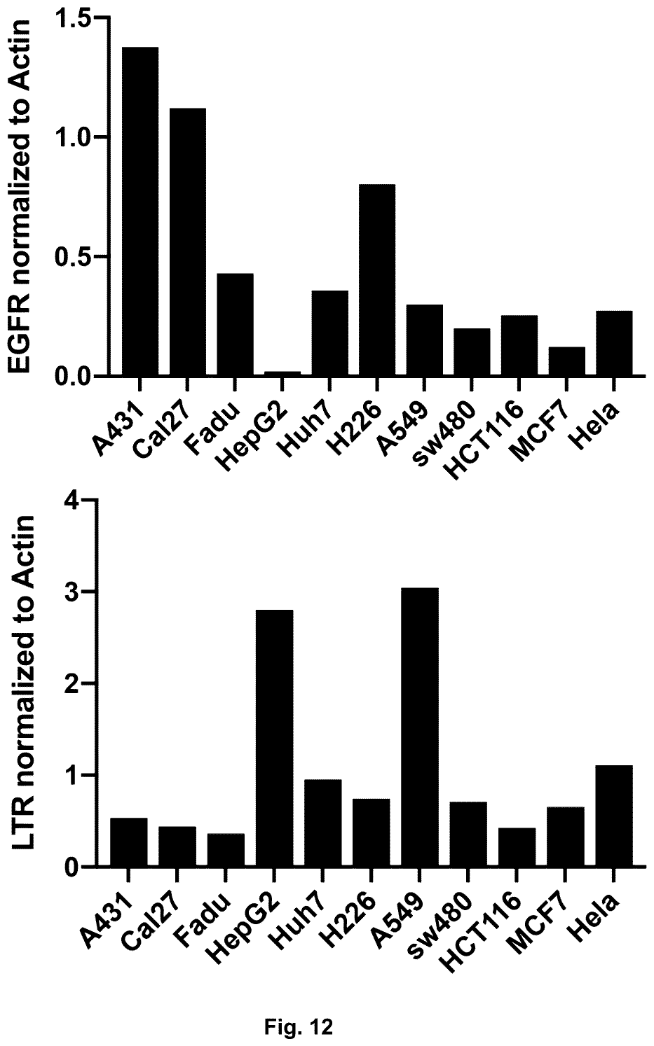

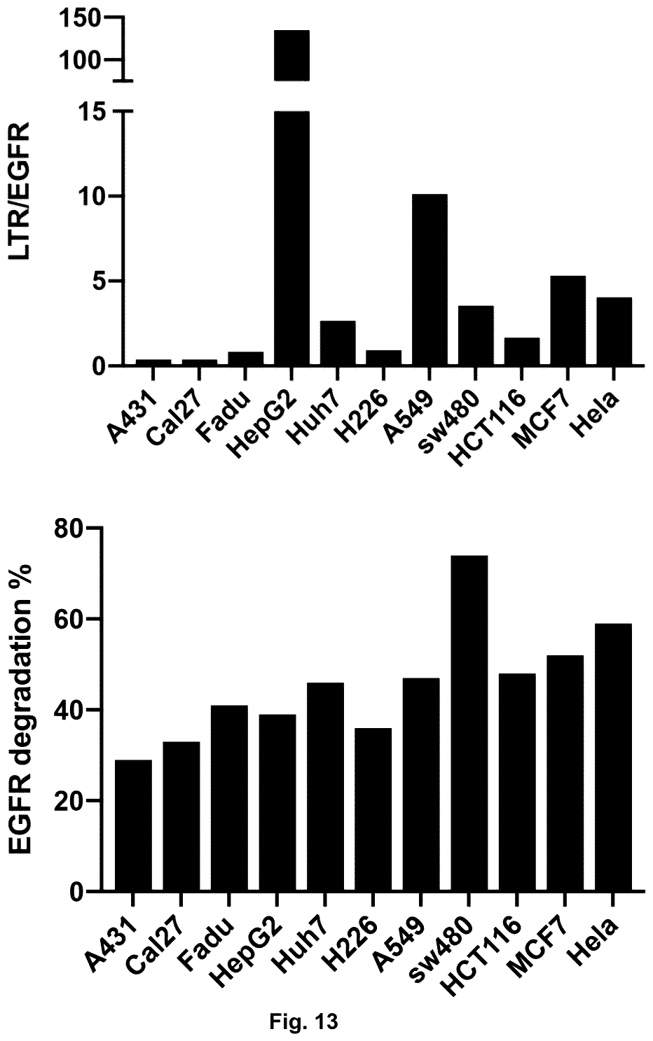

Here we also examined correlation of degradation efficiency and the ratio of LTR (folate receptor)/EGFR expression level. FIG. 11 shows expression levels of EGFR and LTR in multiple cell lines treated with 10 nM Ctx-FA for 48 h. Based on the gel image of FIG. 11, expression levels of EGFR and LTR were normalized to actin (FIG. 12). Percentage of EGFR degradation and LTR/EGFR ratio were calculated (FIG. 13), but no obvious correlation was found between the degradation percentage of EGFR and the ratio of LTR/EGFR.

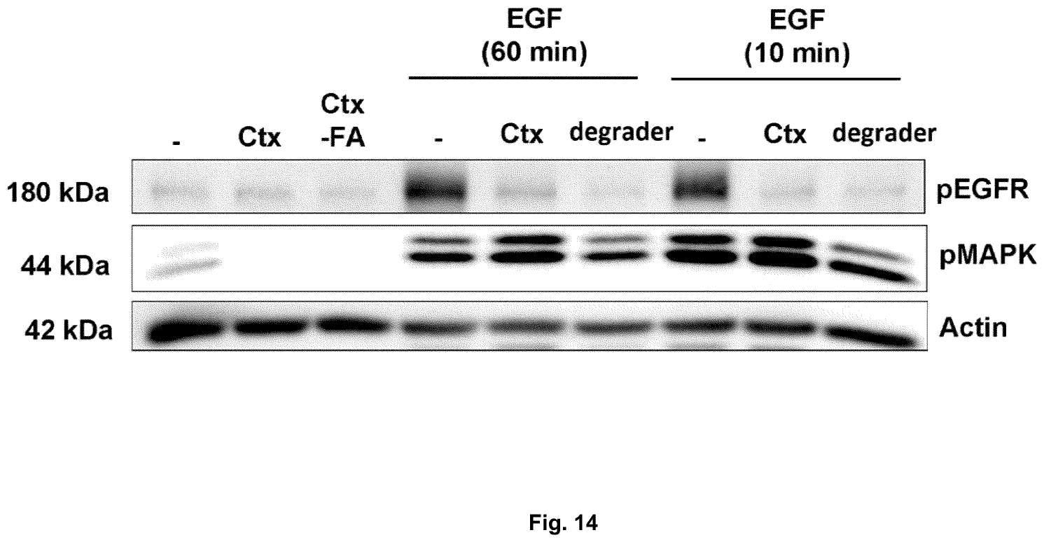

The effect of the Ctx-FA degrader was also tested by EGFR signaling. Cells were pretreated with 10 nM Ctx-FA for 24 h followed by 10 min or 60 min incubation with 100 ng/ml EGF to stimulate the EGFR signaling pathway. Protein was collected for western blotting for phosphorylated EGFR (pEGFR) and MAPK (pMAPK). The results showed very little pEGFR detected in cells treated with Ctx or the Ctx-FA degrader (FIG. 14), indicating that both Ctx and Ctx-FA effectively blocked signaling of EGFR.

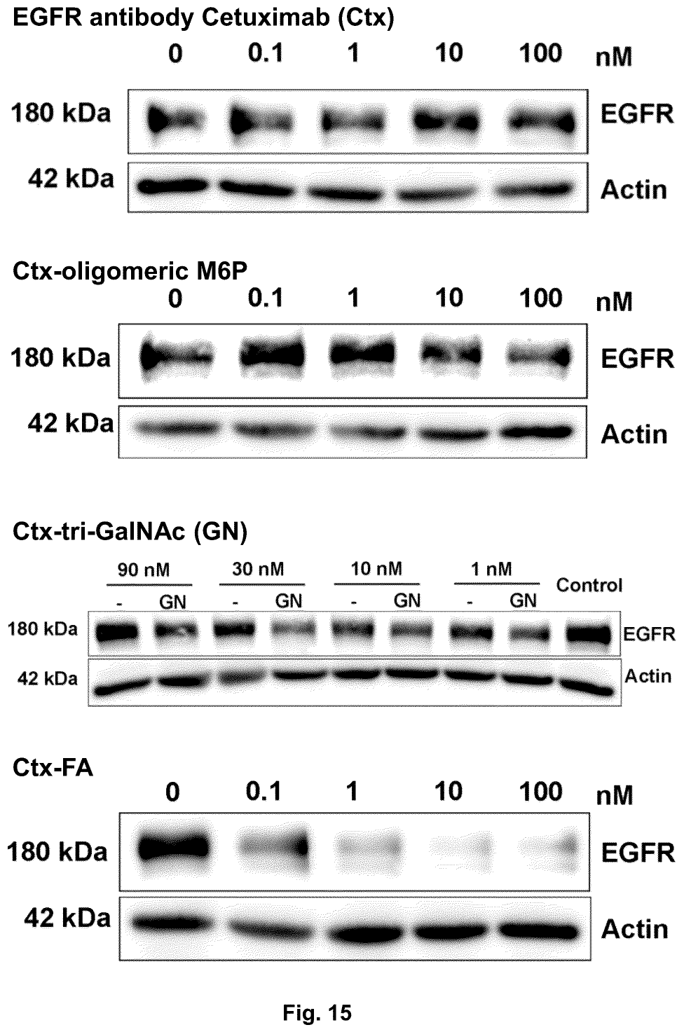

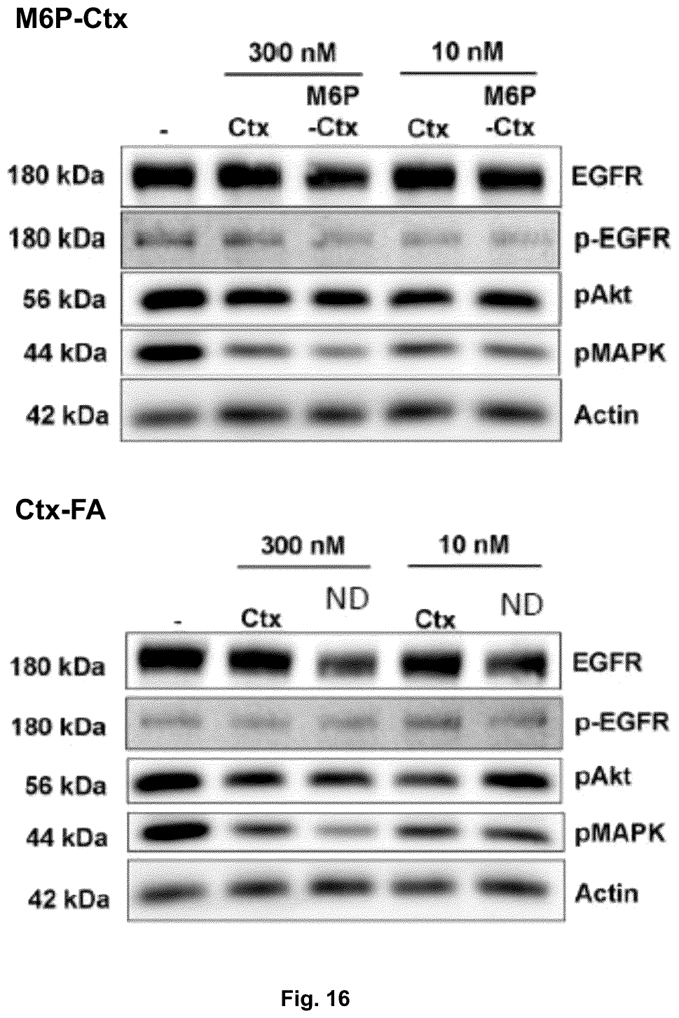

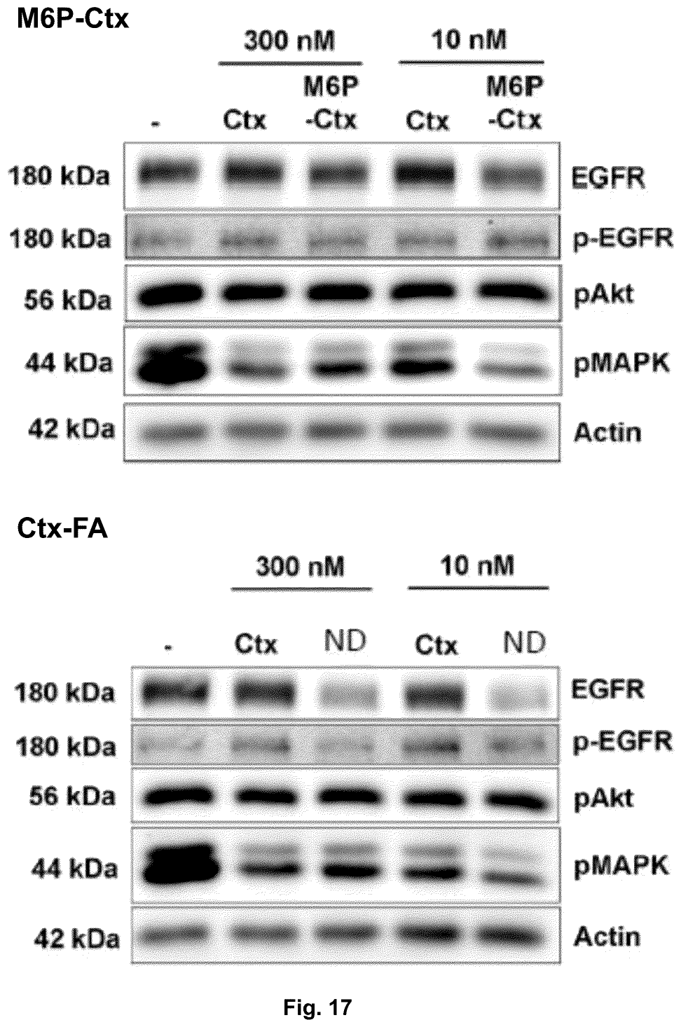

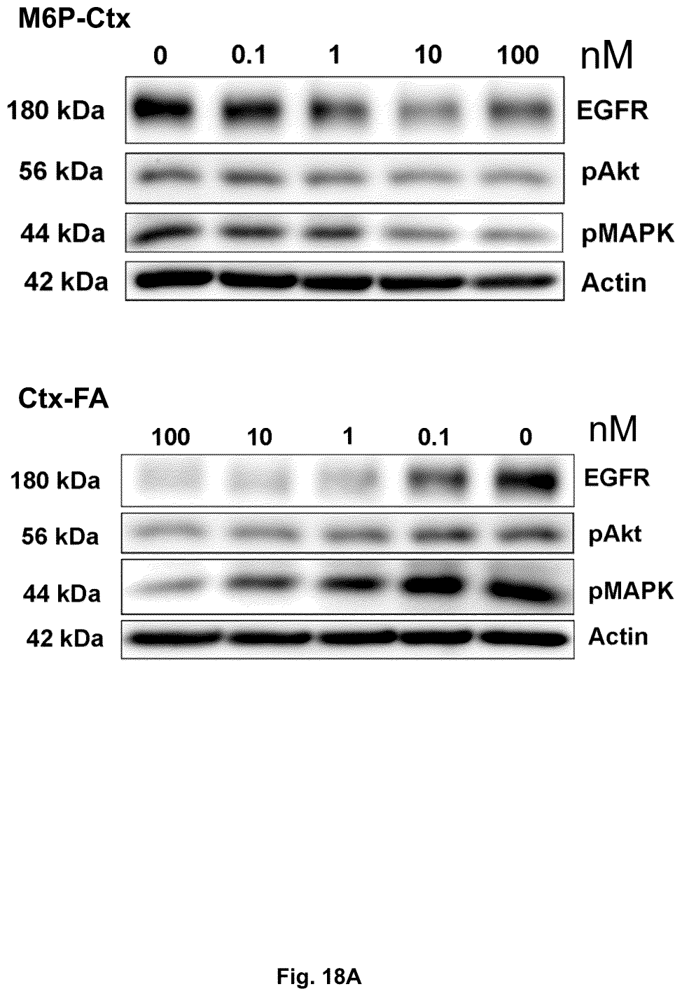

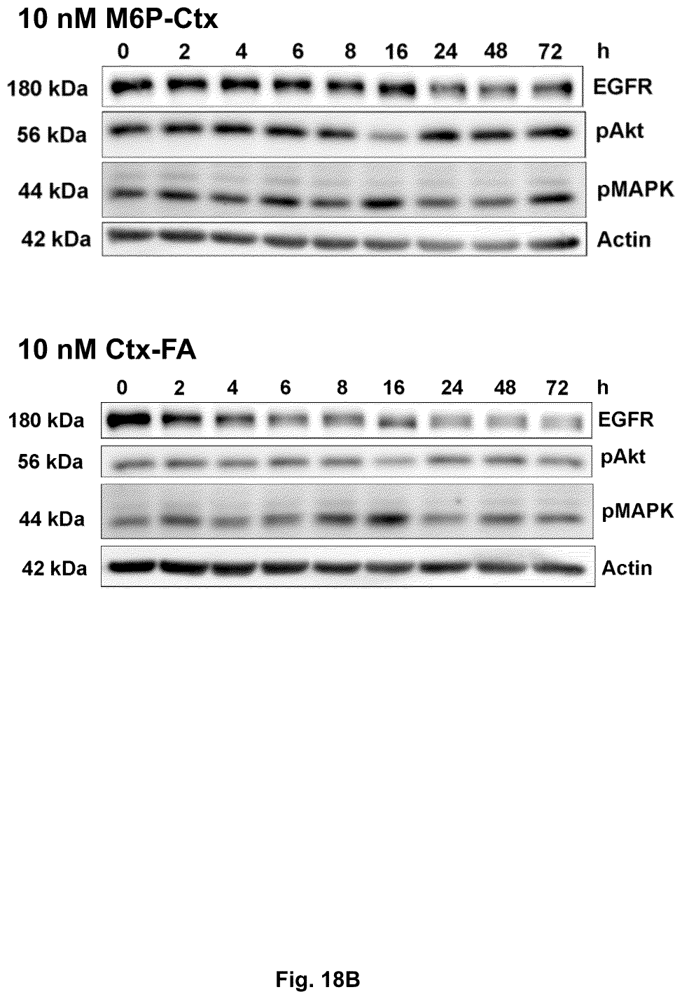

Here we also compared degradation efficiency of the Ctx-FA degrader to other EGFR degraders. As shown in FIG. 15, HepG2 liver cells treated with the Ctx-FA degrader showed superior degradation of EGFR compared to degraders derived from three LTRs, including Ctx alone, Ctx-oligomeric M6P, and Ctx-tri-GalNAc (GN). The Ctx-FA degrader also shows better degradation efficiency of EGFR compared to M6P-Ctx in Cal27 head neck cancer cell (FIG. 16), Fadu head neck cancer cell (FIG. 17) and Hela cell (FIGS. 18A and 18B)

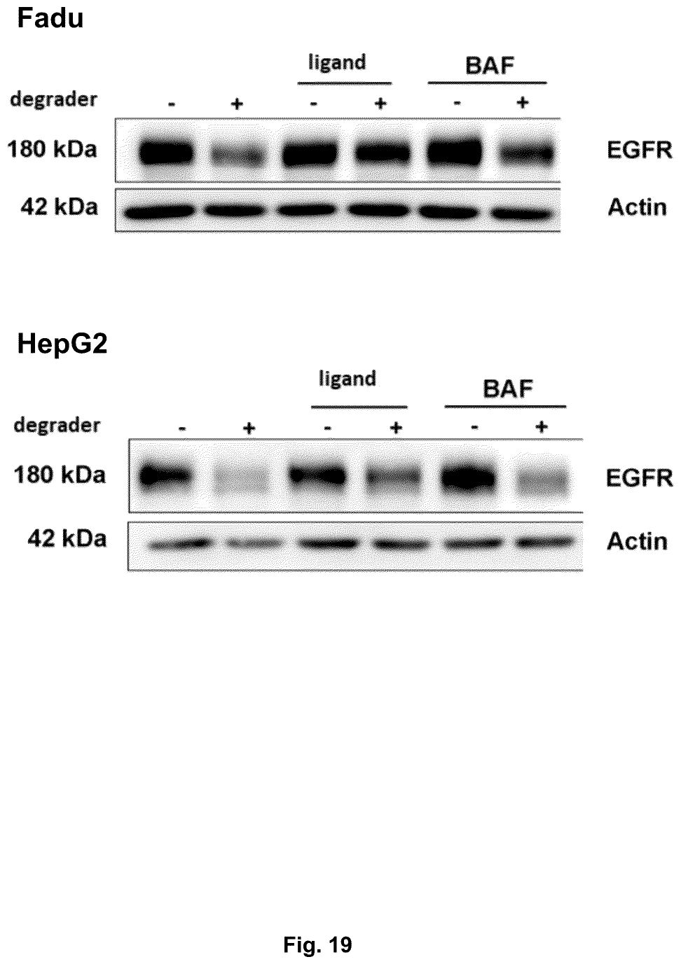

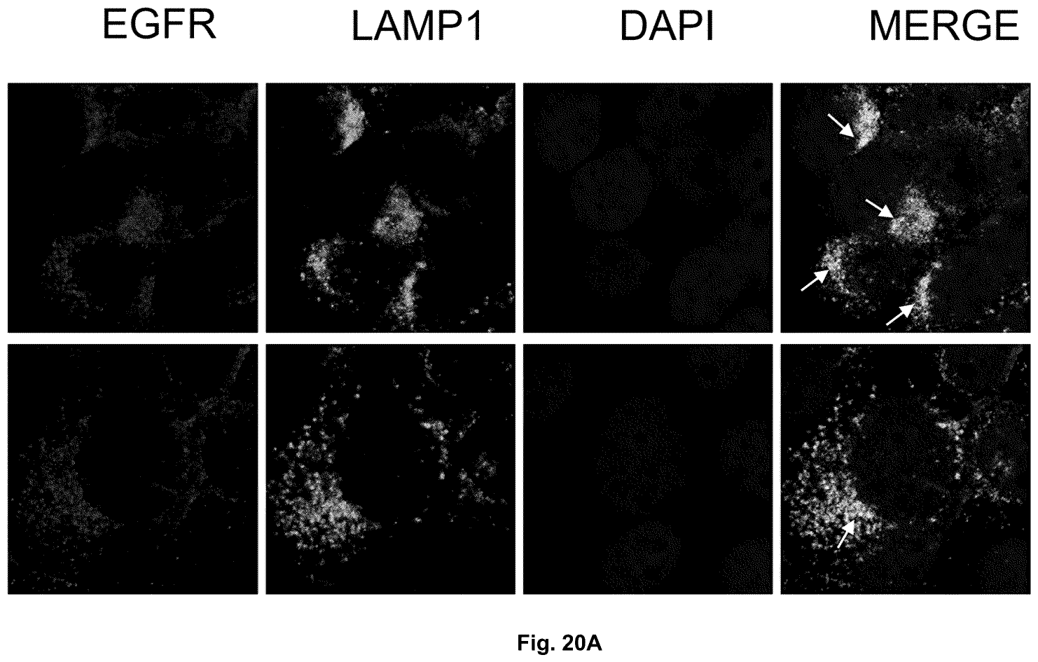

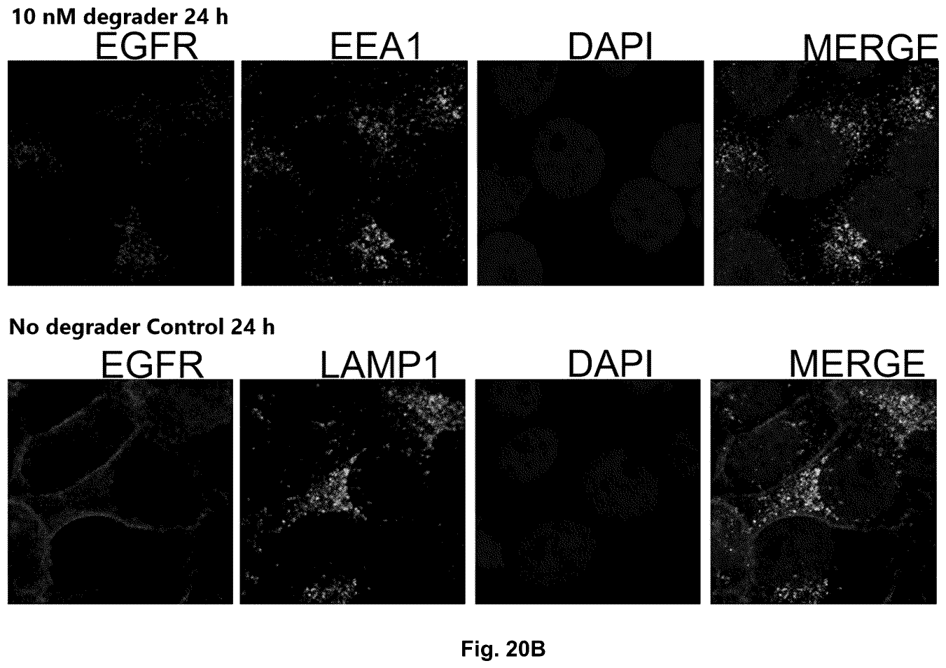

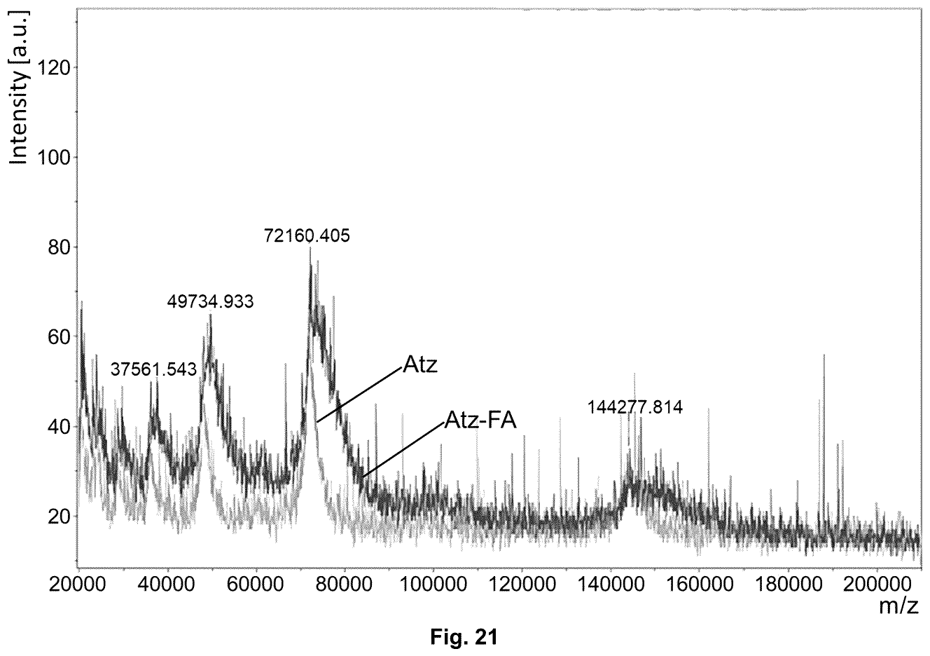

To study the mechanism of the Ctx-FA degrader, Fadu and HepG2 cells were co-treated with the Ctx-FA degrader and lysosome inhibitors folic acid and bafilomycin (BAF). The co-treatment effectively reduced degradation of EGFR (FIG. 19), indicating that degradation of EGFR in presence of the Ctx-FA degrader is via the lysosomal degradation pathway. Colocalization of EGFR with marker for lysosome membrane (LAMP1) was observed in Fadu head neck cancer cells treated with 10 nM Ctx-FA degrader for 24 h, as shown by the confocal microscopy imaging in FIG. 20A, while EGFR localizes on cell membrane in the absence of the Ctx-FA degrader as shown by the confocal microscopy imaging in FIG. 20B.

### Example 3: Degradation of Other Cancer-Relevant Targets (PD-L1 and CD47) Via the Cancer-Specific LTR

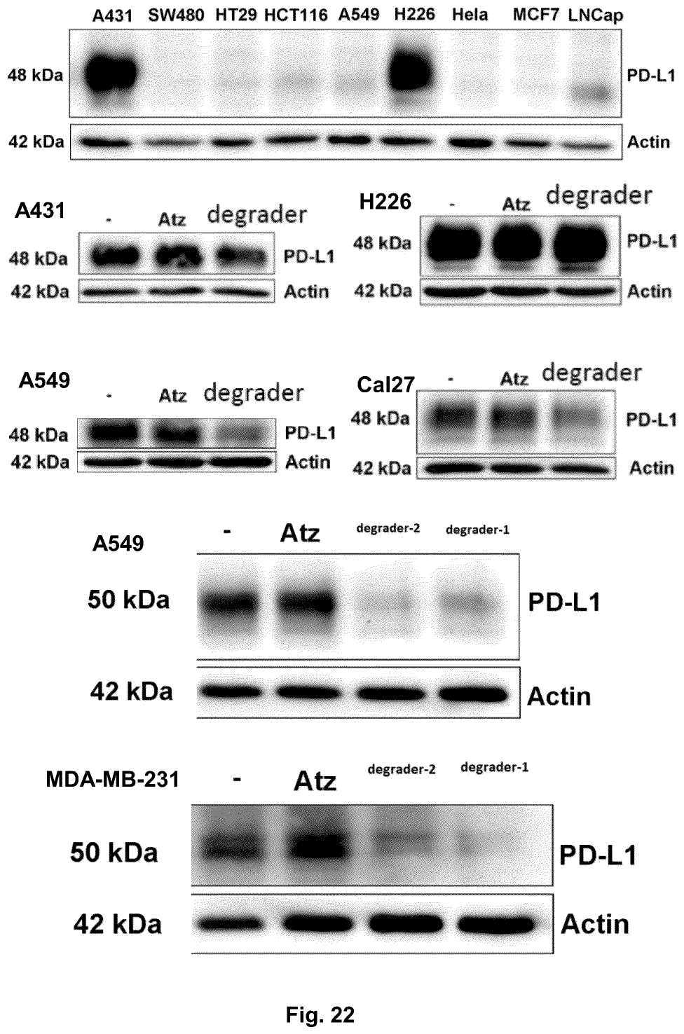

PD-L1 degrader is based on the first FDA approved PD-L1 antibody Atezolizumab (Atz). Atezolizumab is a commercially available monoclonal antibody sold under the brand name “TECENTRIQ”® (Genentech, San Francisco, USA;) FIG. 21 shows the MALDI-MS spectra of Atz with or without labeling of folate. We examined degradation of PD-L1 in multiple cancer cell lines treated with or without folate labeled Atz degrader. Results showed effective degradation of PD-L1 by the folate labeled Atz degrader in most of the tested cells (FIG. 22).

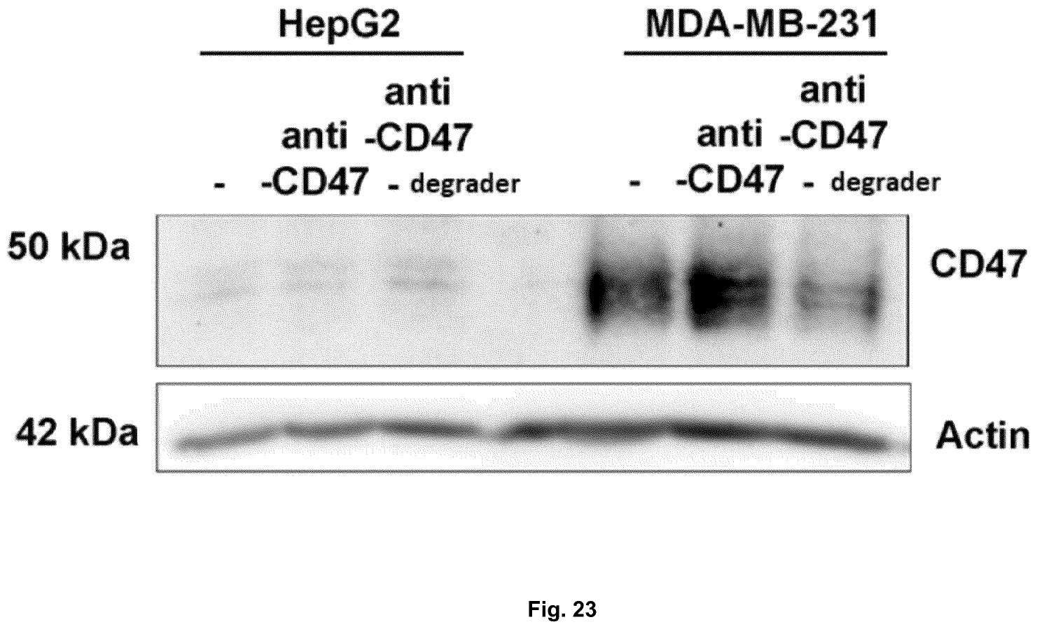

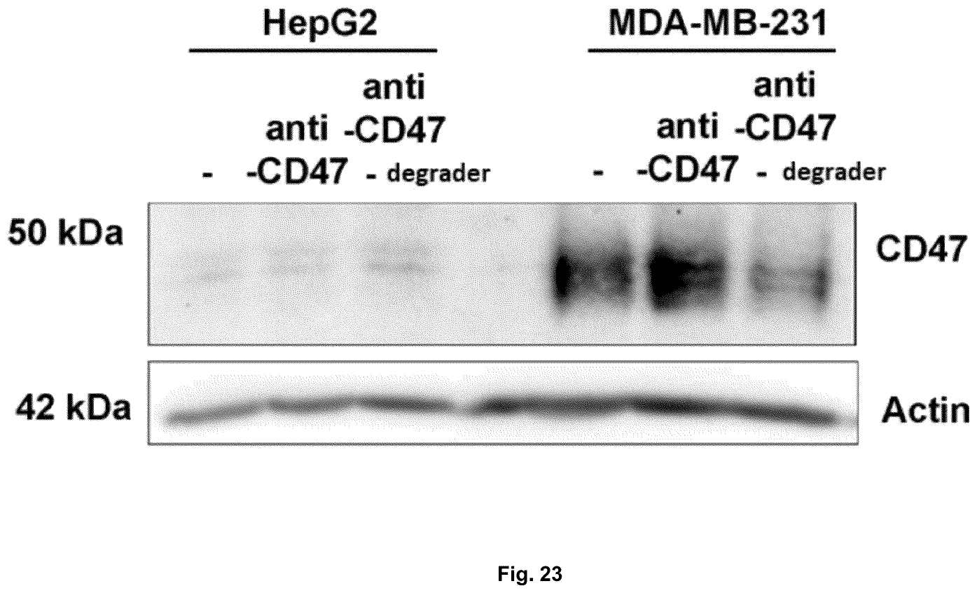

We also explored CD47 degrader based on a CD47 antibody labeled by folate. FIG. 23 shows degradation of CD47 in HepG2 and MDA-MB-231 cells treated with the degrader.

What is claimed is:

1. A bifunctional lysosomal targeting degrader, comprising:
a folate moiety or an analog thereof configured to bind to folate receptor as a shuttle molecule for lysosome degradation; operationally linked to
a protein-binding moiety configured to bind a membrane or extracellular protein.

2. The bifunctional lysosomal targeting degrader of claim 1, wherein the protein-binding moiety is configured to bind a membrane protein.

3. The bifunctional lysosomal targeting degrader of claim 2, wherein the protein-binding moiety is configured to bind a membrane receptor.

4. The bifunctional lysosomal targeting degrader of claim 3, wherein the protein-binding moiety is configured to bind epidermal growth factor receptor (EGFR).

5. The bifunctional lysosomal targeting degrader of claim 2, wherein the protein-binding moiety is configured to bind an immune inhibitory receptor.

6. The bifunctional lysosomal targeting degrader of claim 2, wherein the protein-binding moiety is configured to bind a ligand of an immune inhibitory receptor.

7. The bifunctional lysosomal targeting degrader of claim 6, wherein the protein-binding moiety is configured to bind CD47.

8. The bifunctional lysosomal targeting degrader of claim 2, wherein the protein-binding moiety is configured to bind an immune checkpoint molecule.

9. The bifunctional lysosomal targeting degrader of claim 8, wherein the protein-binding moiety is configured to bind programmed cell death ligand 1 (PD-L1).

10. The bifunctional lysosomal targeting degrader of claim 1, wherein the protein-binding moiety is configured to bind an extracellular protein.

11. The bifunctional lysosomal targeting degrader of claim 1, wherein the protein-binding moiety is selected from a polypeptide, a ligand, an aptamer, a nanoparticle, and a small molecule.

12. The bifunctional lysosomal targeting degrader of claim 1, wherein the protein-binding moiety is a non-protein small molecule.

13. The bifunctional lysosomal targeting degrader of claim 1, wherein the protein-binding moiety is a polypeptide.

14. The bifunctional lysosomal targeting degrader of claim 1, wherein the protein-binding moiety is an antibody.

15. The bifunctional lysosomal targeting degrader of claim 14, wherein the antibody is configured to bind an EGFR protein.

16. The bifunctional lysosomal targeting degrader of claim 15, wherein the antibody is Cetuximab.

17. The bifunctional lysosomal targeting degrader of claim 14, wherein the antibody is configured to bind to a CD47 protein.

18. The bifunctional lysosomal targeting degrader of claim 14, wherein the antibody is configured to bind to a PD-L1 protein.

19. The bifunctional lysosomal targeting degrader of claim 18, wherein the antibody is Atezolizumab.

20. The bifunctional lysosomal targeting degrader of claim 1, further comprising a linker connecting the folate moiety or analog thereof to the protein-binding moiety.

21. The bifunctional lysosomal targeting degrader of claim 20, wherein the linker is PEG.

22. The bifunctional lysosomal targeting degrader of claim 20, wherein the linker is PEG and NHS.

23. The bifunctional lysosomal targeting degrader of claim 1, wherein the bifunctional lysosomal targeting degrader is configured to selectively target cells that express folate receptors.

24. The bifunctional lysosomal targeting degrader of claim 23, wherein the cells that express folate receptors are cancer cells.

25. A pharmaceutical composition comprising the bifunctional lysosomal targeting degrader of claim 1.

26. The pharmaceutical composition of claim 25, further comprising a pharmaceutically acceptable carrier.

27. A method of degrading a membrane or extracellular protein, comprising:
contacting the membrane or extracellular protein with the bifunctional lysosomal targeting degrader of claim 1;
wherein the lysosomal targeting degrader shuttles the membrane or extracellular protein to lysosomes for degradation.

28. A method comprising administering to an individual in need thereof a therapeutically effective, neoplastic cell growth-inhibiting amount of the pharmaceutical composition of claim 25.

29. The method of claim 28, wherein the individual is a human.

30. The method of claim 28, wherein the individual has cancer.
